SEQUENCE LISTING (18 sequences)

SEQ ID NO: 1 - protein, 565 aa, Lactobacillus gasseri
AVKQDEKAATAVKANTEVKANETSTKSASKDNKAELKGQIKDIVKESGVDTSKLTDDQINELNKISFSKEAKSGTQLTYSDFKKIAKTLIEQDARYAVPFFNASKIKNMPAAKTLDAQTGKVEDLEIWDSWPVQDAKTGYVSNWNGYQLVIGMMGVPNTNDNHIYLLYNKYGDNNFNNWKNAGPIFGLGTPVIQQWSGSATLNKDGSIQLYYTKVDTSDNNTNHQKIASATVYLNLEKNQDKISIAHVDNDHIVFEGDGYHYQTYNQWKKTNKGADNIAMRDAHVIDDKDGNRYLVFEASTGTENYQGADQIYQWLNYGGTNKDNLGDFLQILSNSDIKDRAKWSNAAIGIIKLNNDTKNPGVEKVYTPLISAPMVSDEIERPDVVRLGNKYYLFAATRLNRGSNDDAWMAANKAVGDNVAMIGYVSDNLTHGYVPLNESGVVLTASVPANWRTATYSYYAVPVEGRDDQLLITSYITNRGEVAGKGMHATWAPSFLLQINPDNTTTVLAKMTNQGDWIWDDSSENADMMGVLEKDAPNSAALPGEWGKPVDWDLIGGYNLKPHQ

SEQ ID NO: 2 - protein, 567 aa, Lactobacillus gasseri
DAVKQDEKAATSFKTNTEEKANETSTKTASNDNKAELKGQIKDIVKESDVDTSKLTNDQINELNKINFSKEAKSGTQLTYSDFKKIAKTLIEQDARYAIPFFNASKIKNMPAAKTMDAQTGKVEDLEIWDSWPVQDAKTGYVSNWNGYQLVVGMMGVPNTNDNHIYLLYNKYGDNNFNNWKNAGPIFGLGTPVIQQWSGSATLNKDGSIQLYYTKVDTSDNNTNHQKIASATVYLNLEKDQDKISIAHVDNDHIVFEGDGYHYQTYNQWKKTNKGADNIAMRDAHVIDDKDGNRYLVFEASTGTENYQGADQIYQWLNYGGTNKDNLGDFFQILSNSDIKDRAKWSNAAIGIIKLNNDTKNPGVEKVYTPFISSPMVSDEIERPDVVRLGNKYYLFAATRLNRGSNDDAWMAANKAVGDNVAMIGYVSDNLTHGYVPLNESGVVLTASVPANWRTATYSYYAVPVEGRDDQLLITSYITNRGEVAGKGMHATWAPSFLLQINPDNTTTVLAKMTNQGDWIWDDTSENDDMMGVLKKDAPNSAALPGEWGKPVDWDLIGGYNLKPHQP

SEQ ID NO: 3 - protein, 566 aa, Lactobacillus johnsonii
DDVKQVEKKDSVDKTNAEENKDSSVKPAENATKAELKGQVKDIVEESGVDTSKLTNDQINELNKINFSKEAKSGTQLTYNDFKKIAKTLIEQDARYAIPFFNASKIKNMPAAKTLDAQSGKVEDLEIWDSWPVQDAKTGYVSNWNGYQLVIGMMGVPNVNDNHIYLLYNKYGDNDFNHWKNAGPIFGLGTPVIQQWSGSATLNKDGSIQLYYTKVDTSDNNTNHQKLASATVYLNLEKDQDKISIAHVDNDHIVFEGDGYHYQTYDQWKETNKGADNIAMRDAHVIDDDNGNRYLVFEASTGTENYQGDDQIYQWLNYGGTNKDNLGDFFQILSNSDIKDRAKWSNAAIGIIKLNDDVKNPSVAKVYSPLISAPMVSDEIERPDVVKLGNKYYLFAATRLNRGSNDDAWMATNKAVGDNVAMIGYVSDNLTHGYVPLNESGVVLTASVPANWRTATYSYYAVPVEGRDDQLLITSYITNRGEVAGKGMHATWAPSFLLQINPDNTTTVLAKMTNQGDWIWDDSSENPDMMGVLEKDAPNSAALPGEWGKPVDWDLIGGYNLKPHQP

SEQ ID NO: 4 - protein, 663 aa, Lactobacillus reuteri
DTNIENNDSSTVQVTTGDNDIAVKSVTLGSGQVSAASDTTIRTSANANSASSAANTQNSNSQVASSAAITSSTSSAASSNNTDSKAAQENTNTAKNDDTQKAAPANESSEAKNEPAVNVNDSSAAKNDDQQSSKKNTTAKLNKDAENVVKKAGIDPNSLTDDQIKALNKMNFSKAAKSGTQMTYNDFQKIADTLIKQDGRYTVPFFKASEIKNMPAATTKDAQTNTIEPLDVWDSWPVQDVRTGQVANWNGYQLVIAMMGIPNQNDNHIYLLYNKYGDNELSHWKNVGPIFGYNSTAVSQEWSGSAVLNSDNSIQLFYTRVDTSDNNTNHQKIASATLYLTDNNGNVSLAQVANDHIVFEGDGYYYQTYDQWKATNKGADNIAMRDAHVIEDDNGDRYLVFEASTGLENYQGEDQIYNWLNYGGDDAFNIKSLFRILSNDDIKSRATWANAAIGILKLNKDEKNPKVAELYSPLISAPMVSDEIERPNVVKLGNKYYLFAATRLNRGSNDDAWMNANYAVGDNVAMVGYVADSLTGSYKPLNDSGVVLTASVPANWRTATYSYYAVPVAGKDDQVLVTSYMTNRNGVAGKGMDSTWAPSFLLQINPDNTTTVLAKMTNQGDWIWDDSSENLDMIGDLDSAALPGERDKPVDWDLIGYGLKPHD

SEQ ID NO: 5 - protein, 422 aa, Bacillus agaradhaerens
TSDWDAEDDYTAVWTRQQAENVALTKDTTAPLLETDEDFELVAPDKWVWDTWPLQNRDGSLAQVNGYTIAFALVAPRDLGWGERHTEARIGMFYSKDGKDWTYAGIPYDYDKAYGHMQWAGSAMLDKDGKVHFFYTATGRKDNSEYFDQPGWEPMAEQRLAKTTFDISADKDGVHLTKEDEHQIMLEADGEYYETLGQWGSNGNIISAFRDPFFFQDPNTGEEYIIWEGQAGPKSNGLKPENIGDEAYRKNANVPDRAELYNGNIGIAKVLDEDVSELKMLPPLLESIGVNHQLERPHVVVDGDTYYLLTISHTFTYAPGLTGPEGLYGFVNEGGLRGDYEPLNDGGLVIGNPAESPGQAYSWWVAPDGQVISFINEPLDENGEVQFVGTFAPTLQLSFDGDQTKIEKEMGYGEIRPFGAYR

SEQ ID NO: 6 - protein, 663 aa, Lactobacillus reuteri
DTNTENNDSSTVHVTTGDNDIAVKSAILGSGQVSAASDATIKNSANANSASSAANTQNSNSQVASSAATTSSTSSAASSNNTDSKAAQENANTAKNDDTQKAAPANESSEAKNEPAVNVNDSSAAKNDDQQSSKKNTTAKLNKDAENVVKKAGIDPNSLTDDQIKALNKMNFSKAAKSGTQMTYNDFQKIADTLIKQDGRYTVPFFKASEIKNMPAATTKDAQTNTIEPLDVWDSWPVQDVRTGQVANWNGYQLVIAMMGIPNQNDNHIYLLYNKYGDNELSHWKNAGPIFGYNSTAVSQEWSGSAVLNSDNSIQLFYTRVDTSDNNTNHQKIASATLYLTDNNGNVSLAQVANDHIVFEGDGYYYQTYDQWKATNKGADNIAMRDAHVIEDDNGDRYLVFEASTGLENYQGENQIYNWLNYGGDDAFNIKSLFRILSNDDIKSRATWANAAIGILKLNKDEKNPKVAELYSPLISAPMVSDEIERPNVVKLGNKYYLFAATRLNRGSNDDTWMNANYAVGDNVAMVGYVADSLTGSYKPLNDSGVVLTASVPANWRTATYSYYAVPVAGKDDQVLVTSYMTNRNGVAGKGMDSTWAPSFLLQINQDNTTTVLAKMTNQGDWIWDDSSENLDMIGDLDSAALPGERDKPVDWDLIGYGLKPHD

SEQ ID NO: 7 - protein, 635 aa, Aspergillus aculeatus
SYHLDTTAPPPTNLSTLPNNTLFHVWRPRAHILPAEGQIGDPCAHYTDPSTGLFHVGFLHDGDGIAGATTANLATYTDTSDNGSFLIQPGGKNDPVAVFDGAVIPVGVNNTPTLLYTSVSFLPIHWSIPYTRGSETQSLAVARDGGRRFDKLDQGPVIADHPFAVDVTAFRDPFVFRSARLDVLLSLDEEVARNETAVQQAVDGWTEKNAPWYVAVSGGVHGVGPAQFLYRQNGGNASEFQYWEYLGEWWQEATNSSWGDEGTWAGRWGFNFETGNVLFLTEEGHDPQTGEVFVTLGTEGSGLPIVPQVSSIHDMLWAAGEVGVGSEQEGAKVEFSPSMAGFLDWGFSAYAAAGKVLPASSAVSKTSGVEVDRYVSFVWLTGDQYEQADGFPTAQQGWTGSLLLPRELKVQTVENVVDNELVREEGVSWVVGESDNQTATLRTLGITIARETKAALLANGSVTAEEDRTLQTAAVVPFAQSPSSKFFVLTAQLEFPASARSSPLQSGFEILASELERTAIYYQFSNESLVVDRSQTSAAAPTNPGLDSFTESGKLRLFDVIENGQEQVETLDLTVVVDNAVVEVYANGRFALSTWARSWYDNSTQIRFFHNGEGEVQFRNVSVSEGLYNAWPERN

SEQ ID NO: 8 - protein, 682 aa, Aspergillus sydowii
MKLPSSLDILLARQAVGGTEVDYDSPPPDLTTLPENSLFETWRPKIHVLPPNGQIGDPCAHYNDPATGLFHVGFLHNGTGISSVYTDDLVTYRDINPNGGYIIVAGGPNDPEAVFDGSVIPSGIDDLPTLLYTSVTSLPIHWTLPYTPGSETQSLAVSDDGGHHFDKLDRGPVIPLPPDGLDVTAFRDPYVFQNHEVDEVTGSDPDTWYAAISGGVHDVGPGIFLYRNQDSSFENWEYLGEWWQEPANSTWGDGTWAKRWGYNFETGNVFSLDREGYNVDGHTFMTIGVEGAYAPIQPSVTSMHAMLWAAGNVSSENGENVTFTPYMAGALDWGMAAYAGAGKVLPSTSQASEKSGAPDRFISWVWLTGDEFGAAAGFPAAQQGWQNTLLLPRELSIHTIQNVVDNELIHETASWRVAEHGGERRSGGVELETLGINIARETYDAIVSSGTSFEEPSRDINESGTIPFERSPTSRFFALEAQISFPQSARDSEVQSGFQILASELEWTTIYYQFSNESIVIDRNHTSAASETTPGLGTVTESGRIRLFDIAGGCDHDGHGGHDGGNDDDHNGDGDHSGDGDHNDDDDHNVDGDDKERARYQKRDGPCDKDHDKVETLDLTIVVDNSVLEVYANSRFVVSTWVRPWYTNSTEIRFFHNGEGEVSFDNIAVHDGLYDAYPDRDN

SEQ ID NO: 9 - protein, 443 aa, Bacillus amyloliquefaciens
KENNQKAYKETYGVSHITRHDMLQIPKQQQNEKYQVPQFDQSTIKNIESAKGLDVWDSWPLQNADGTVAEYNGYHVVFALAGSPKDADDTSIYMFYQKVGDNSIDSWKNAGRVFKDSDKFDANDPILKDQTQEWSGSATFTSDGKIRLFYTDYSGKHYGKQSLTTAQVNVSKSDDTLKINGVEDHKTIFDGDGKTYQNVQQFIDEGNYTSGDNHTLRDPHYVEDKGHKYLVFEANTGTENGYQGEESLFNKAYYGGGTNFFRKESQKLQQSAKKRDAELANGALGIIELNNDYTLKKVMKPLITSNTVTDEIERANVFKMNGKWYLFTDSRGSKMTIDGINSNDIYMLGYVSNSLTGPYKPLNKTGLVLQMGLDPNDVTFTYSHFAVPQAKGNNVVITSYMTNRGFFEDKKATFGPSFLMNIKGNKTSVVKNSILEQGQLTVN

SEQ ID NO: 10 - protein, 455 aa, Bacillus megaterium
KGNDSKDFNNSYGISHITRDNMVKIPQQQNSDQFKVPAFDESTIKNIASAKGKNASGNTIDLDVWDSWPLQNADGTVATYHGYQIVFALAGDPKDSNDTSVYLFYKKAGDKSIDSWKNAGRVFKDSDKFVPNDPHLKNQTQEWSGSGTLTKDGKVRLFYTDYSGKQYGKQTLTTAQVNMSQPNDNTLKVDGVEDYKSIFDGDGKIYQTVQQFIDEGGYDTGDNHTLRDPHYIEDNGHKYLVFEANTGTEDGYQGEDSLYNRAYYGGNNPFFQSEKKKLLEGSNKEKASLANGALGIIELNDDYTLKKVMKPLITSNTVTDEIERANIFKKDGKWYLFTDSRGSKMTIDGIGQDDVYMLGYVSNTLTGKYKPLNDTGLVLHMDLDPNDKTFTYSHFAVPQTKGDNVVITSYMTNRGFYEDNHSTFAPSFLVNIDGSKTSVVKDRVLEQGQLTVDED

SEQ ID NO: 11 - protein, 4 aa, Artificial Sequence
  Poly-histidine tag
HHHH

SEQ ID NO: 12 - protein, 6 aa, Artificial Sequence
  Poly-histidine tag
HHHHHH

SEQ ID NO: 13 - protein, 8 aa, Artificial Sequence
  Poly-histidine tag
HHHHHHHH

SEQ ID NO: 14 - protein, 8 aa, Artificial Sequence
  Strep tag
WSHPQFEK

SEQ ID NO: 15 - protein, 8 aa, Artificial Sequence
  FLAG tag
DYKDDDDK

SEQ ID NO: 16 - protein, 9 aa, Artificial Sequence
  Human influenza hemagglutinin (HA) tag
YPYDVPDYA

SEQ ID NO: 17 - protein, 9 aa, Artificial Sequence
  Myc tag
EQKLISEED

SEQ ID NO: 18 - protein, 14 aa, Artificial Sequence
  V5 tag
GKPIPNPLLGLDST

(19) United States
(12) Patent Application Publication
(10) Pub. No.: US 2023/0398191 A1
(43) Pub. Date: Dec. 14, 2023

(54) USE OF FRUCTOSYLTRANSFERASE

(71) Applicant: Inulox Ltd., Thame (GB)

(72) Inventors: Andreas Kjaer, Thame (GB); Niels Wicke, Thame (GB); Carolin Schulte, Thame (GB); Joel Kosmin, Thame (GB); Joshua Sauer, Thame (GB)

(21) Appl. No.: 18/250,593

(22) PCT Filed: Nov. 2, 2021
(86) PCT No.: PCT/GB2021/052834

§ 371 Date: Apr. 26, 2023

(30) Foreign Application Priority Data
Nov. 3, 2020 (GB) 2017421.5

(51) Int. Cl. A61K 38/45; A23G 1/42; A23G 1/40; A61K 9/00

(52) U.S. Cl. CPC A61K 38/45; A23G 1/423; A23G 1/40; A61K 9/0053

(57) ABSTRACT

Provided herein are methods for reducing fructose uptake in a subject; as well as associated compositions. The compositions are useful in therapeutic and non-therapeutic uses such as appetite suppression and treating or preventing metabolic syndrome, obesity, non-alcoholic fatty liver disease, and constipation.

### FIELD

The present disclosure provides methods involving the administration of isolated fructosyltransferases to subjects in order to produce oligofructosaccharides (fructooligosaccharides) in vivo. The disclosure also relates to in vivo methods of reducing fructose uptake in a subject; to in vivo methods of reducing the formation of fructose via metabolism of sucrose in a subject; to nutraceutical compositions comprising isolated fructosyltransferases; and to pharmaceutical compositions comprising isolated fructosyltransferases. Also provided is a food composition comprising an isolated fructosyltransferase as described herein. The compositions are useful in the treatment of conditions such as metabolic syndrome; obesity; and for reducing a subject's appetite. The compositions described herein have application in both therapeutic and non-therapeutic uses.

### BACKGROUND

Sucrose is a disaccharide formed from glucose and fructose monomer units. Sucrose is commonly informally referred to as “sugar”, reflecting the fact that fully refined table sugar comprises around 99.9% sucrose. Sucrose is produced naturally in plants such as sugar cane and sugar beet. Sucrose is commonly added to foodstuffs in order to increase the sweetness of such foods, and also as a preservative. The use of sucrose in foodstuffs, in particular baked goods, is typically considered to be important for satisfactory “mouthfeel” (texture etc). Sucrose is a major commodity with annual global production in the order of hundreds of millions of tonnes.

Once consumed by subjects, typically mammalian subjects, sucrose is typically metabolised into its component monomeric units (glucose and fructose) by enzymes such as sucrases, isomaltase glycoside hydrolases, and invertases, which are often found in (e.g.) the duodenum. The glucose and fructose units thus generated are rapidly absorbed into the bloodstream. Sucrose is a high energy compound yielding around 17 kJ/g.

The significant calorific value of sucrose means that health authorities around the world have recommended limits on daily consumption by subjects such as humans. For example, the UK National Health Service recommends that adults should not consume more than 30 g of sugar a day. Recommended daily limits for children are lower, at approximately 19-24 g per day. It is also recommended that sugar should not exceed more than 5% of the total calories obtained from food and drink per day. Broadly similar guidelines are issued by other health authorities around the world. For example, the US Dietary Guidelines for Americans 2015-2020 recommends limiting sugar intake for adults to around 200 calories (kcal) (ca. 50 g).

Despite these recommendations, typical daily sugar consumption is significantly in excess of the suggested levels. For example, the Dietary Guidelines for Americans indicates that the average American adult consumes approximately 70 g of sugar per day, corresponding to an energy intake of around 270 kcal.

Excess sucrose consumption is problematic as it is associated with numerous health issues. For example, excess sucrose consumption is believed to contribute to development of metabolic syndrome, including increased risk for type 2 diabetes; and to weight gain and obesity in adults and children.

In view of these issues, one strategy that is widely promoted is for subjects to simply reduce their overall sugar intake. This strategy can be successful where circumstances allow. However, for many adults the reality is that consuming high-sugar foods is a pleasurable and often unavoidable part of life. Low-sugar alternatives to high-sugar foods are often perceived as being less desirable, e.g. as being less satisfying. Furthermore, in many cases low-sugar options are simply not available, whether due to scarcity of supply in some regions or through social pressure to partake of high-sugar foods. In addition, for many people the need to consider the sugar levels of foodstuffs is a significant time and mental burden. In practice, such subjects tend to simply consume excess sugar levels leading to health problems as set out above.

These difficulties have been long recognised and various attempts have been made to address them. Most attempts have focussed on reducing sugar levels in commercially available foodstuffs. However, this can have significant adverse implications, in terms of increased production costs, reduced shelf-life requiring the need for artificial preservatives, which have been linked to health and taste issues, and a perceived worsening in taste. For example, the commonly-used artificial sweetener saccharin has been associated with a bitter aftertaste. These issues have led to a degree of consumer resistance to products containing artificial sweeteners.

In view of these difficulties, one approach that has been considered is to treat foodstuffs made using sucrose in order to reduce their calorific burden without the need for artificial sweeteners. One approach that has been described is the industrial treatment of sucrose-containing foods prior to their consumption with enzymes such as glycosyltransferases. These enzymes have been shown to convert sucrose to fructooligosacharides which cannot be metabolised by the body, and thus do not contribute to a subject's calorific intake.

Such methods have shown promise, but significant problems remain. One key issue concerns availability of such foodstuffs: in practice, commercially produced foods may not have been treated in this way and a consumer wishing to limit their sugar intake may have no way of knowing whether a given foodstuff has or has not been so treated. Issue also arises even when foodstuffs pre-treated in this manner are available, as the choice available to consumers is typically limited. Consumers may thus be placed in the position of having to choose between the food they actually want, and an alternative pre-treated product which may be less desirable, e.g. for cost or taste reasons, or for reasons of regarding limited availability. Furthermore, the fructooligosacharides generated in the pretreatment of such food may lead (or be perceived to lead) to adverse effects, such as a worsened taste. In addition, “mouthfeel” is often perceived to be adversely affected by fructooligosaccharides generated in the pretreatment of food, as these can negatively affect the texture of the foodstuffs treated. These difficulties mean that even subjects seeking to make healthy choices often end up in practice consuming high-sucrose foods. These issues particularly affect foodstuffs treated with previously used glycosyltransferases where typically high concentrations of the enzyme are needed in order to achieve useful conversion efficiencies.

Accordingly, there is a need for new and/or improved methods of reducing the problems associated with excess sucrose consumption without compromising on taste or mouthfeel.

### SUMMARY

The present inventors have recognised the issues above. The methods disclosed herein address some or all of such problems.

Accordingly, the present disclosure relates to an in vivo method of reducing fructose uptake in a subject. The method comprises administering to the subject a fructosyltransferase enzyme. The enzyme administered to the subject is an isolated enzyme. The isolated fructosyltransferase converts sucrose to fructooligosacharides thus preventing or reducing generation of free fructose by in vivo metabolism of sucrose in the subject. The reduction or prevention of free fructose reduces or prevents fructose uptake by the subject.

Accordingly, the present disclosure provides an in vivo method of reducing fructose uptake in a subject, the method comprising administering to the subject an isolated fructosyltransferase.

Also provided is an in vivo method of reducing the formation of fructose via metabolism of sucrose in a subject, the method comprising administering to the subject an isolated fructosyltransferase.

Also provided is an in vivo method of reducing glucose uptake and/or of reducing the formation of glucose via metabolism of sucrose in a subject, the method comprising administering to the subject an isolated fructosyltransferase.

Typically, the method is a method of producing a fructooligosacharide in a subject in vivo, comprising administering to the subject an isolated fructosyltransferase and thereby converting sucrose to said fructooligosacharide.

Typically, said fructosyltransferase is an inulosucrase or a levansucrase.

In one embodiment, the fructosyltransferase is an inulosucrase of EC class 2.4.1.9. Accordingly, the method is typically a method of reducing fructose uptake in a subject and producing inulin in vivo, the method comprising administering to the subject an isolated inulosucrase and thereby converting sucrose to inulin in vivo. The method may be a method of reducing the formation of fructose via metabolism of sucrose in a subject and producing inulin in vivo, comprising administering to the subject an isolated inulosucrase and thereby converting sucrose to inulin in vivo. In another embodiment, the fructosyltransferase is a levansucrase of EC class 2.4.1.10.

Typically, the fructosyltransferase comprises a polypeptide according to any one of SEQ ID NOs: 1 to 10 or a functional variant thereof.

Typically, said fructosyltransferase has:
i) at least 70% homology to SEQ ID NO: 1, wherein said homology is assessed relative to positions 128, 129, 153, 158, 159, 160, 162, 196, 197, 281, 282, 298, 379, 381, 399, 402, 457, 458 and 480 of SEQ ID NO: 1; or
ii) at least 70% homology to SEQ ID NO: 5, wherein said homology is assessed relative to positions 49, 50, 73, 82, 83, 84, 85, 86, 119, 120, 209, 210, 293, 295 and 361 of SEQ ID NO: 5; or
iii) at least 70% homology to SEQ ID NO: 8, wherein said homology is assessed relative to positions 54, 55, 56, 57, 58, 59, 74, 75, 116, 265, 338, 339, 366, 370, 372 and 373 of SEQ ID NO: 8.

Often, the fructosyltransferase comprises alanine at the position corresponding to A182 of SEQ ID NO: 1. Sometimes, said fructosyltransferase comprises phenylalanine at the position corresponding to F372 of SEQ ID NO: 8 and/or comprises glycine at the position corresponding to G373 of SEQ ID NO: 8.

Typically, the fructosyltransferase has a solubility GRAVY score of −0.4 or more negative than −0.4.

Usually, the fructosyltransferase is derived from an organism of genus Lactobacillus, Bacillus, Leuconostoc, Streptomyces, Aspergillus, or Clostridium. Typically, said fructosyltransferase is derived from an organism of species Lactobacillus gasseri, Lactobacillus johnsonii, Lactobacillus reuteri, Bacillus agaradhaerens, Bacillus amyloliquefaciens, Bacillus megaterium, Bacillus subtilis, Leuconostoc citreum, Leuconostoc mesenteroides, Streptomyces viridochromogenes, Aspergillus acelatus, Aspergillus sydowii, or Clostridium acetobutylicum.

Typically, the fructosyltransferase is expressed by or is obtainable by expression from an organism of genus Escherichia, Lactobacillus, Saccharomyces, Bacillus, Pichia, Trichoderma or Aspergillus, preferably E. coli, S. cerevisiae, B. subtilis, P. pastoris, T reesei, A. niger, or A. oryzae.

Often, the fructosyltransferase is comprised in a nutraceutical composition comprising said fructosyltransferase and one or more nutraceutically acceptable filler, stabilizing agent, coloring agent or flavouring agent. Typically, said nutraceutical composition is formulated as a tablet, a troche, a lozenge, an aqueous or oily suspension, a dispersible powder or as granules. Usually, said method comprises orally administering said fructosyltransferase or said nutraceutical composition to said subject.

Typically, such methods are non-therapeutic methods. Typically said methods do not comprise the treatment of the human or animal body by therapy or surgery.

Also provided is an isolated fructosyltransferase for use in
i) reducing fructose uptake in a subject;
ii) reducing the formation of fructose via metabolism of sucrose in a subject;
iii) reducing glucose uptake and/or of reducing the formation of glucose via metabolism of sucrose in a subject;
iv) producing a fructooligosacharide in a subject, said use comprising administering to the subject an isolated fructosyltransferase and thereby converting sucrose to said fructooligosacharide;

Typically:
i) said isolated fructosyltransferase is for use in reducing fructose uptake and producing inulin in the subject, and said use comprises administering to the subject the isolated inulosucrase and thereby converting sucrose to inulin in vivo; or
ii) said isolated fructosyltransferase is for use in reducing the formation of fructose via metabolism of sucrose in the subject and producing inulin in vivo, and said use comprises administering to the subject the isolated inulosucrase and thereby converting sucrose to inulin in vivo.

Typically, in said uses, said fructosyltransferase is as defined herein. Typically, said uses comprise orally administering said isolated fructosyltransferase to said subject. Said uses may comprise orally administering said isolated fructosyltransferase to said subject in the form of a pharmaceutically acceptable composition or a nutraceutically acceptable composition.

Also provided is a nutraceutical composition comprising an isolated fructosyltransferase and one or more nutraceutically acceptable filler, stabilizing agent, colouring agent or flavouring agent. Typically, said composition is a dietary supplement.

Further provided is a pharmaceutically acceptable composition comprising an isolated fructosyltransferase and one or more pharmaceutically acceptable carrier, excipient, or diluent.

In the provided compositions, the isolated fructosyltransferase is typically an isolated fructosyltransferase described herein. Typically, the provided compositions are for oral administration. Typically, a provided composition comprises an enteric coating. Often, a provided composition is formulated as a tablet, a troche, a lozenge, an aqueous or oily suspension, a dispersible powder or as granules.

Also provided herein a composition as described herein for use in medicine.

Also provided is a food composition or foodstuff comprising an isolated fructosyltransferase and one or more carbohydrate, fat, lipid, flavouring agent, or colouring agent. The food composition or foodstuff may comprise sucrose. The isolated fructosyltransferase is typically as defined herein.

Also provided herein is a method of suppressing a subject's appetite, comprising administering to the subject an isolated fructosyltransferase or a composition as described herein. Typically, the isolated fructosyltransferase is as defined herein. Typically, the method is a non-therapeutic method. Typically said method does not comprise the treatment of the human or animal body by therapy or surgery. Typically, said method comprises orally administering said isolated fructosyltransferase or composition to said subject.

Still further provided is an isolated fructosyltransferase, or a pharmaceutically acceptable composition as described herein, for use in treating or preventing metabolic syndrome, diabetes, non-alcoholic fatty liver disease or constipation in a subject in need thereof. Also provided is an isolated fructosyltransferase, or a pharmaceutically acceptable composition as described herein for use in treating or preventing obesity in a subject in need thereof. Typically, such uses comprise orally administering said isolated fructosyltransferase or said pharmaceutically acceptable composition to said subject. Typically the isolated fructosyltransferase is as defined herein.

### BRIEF DESCRIPTION OF THE FIGURES

FIG. 1. Activity screen of fructosyltransferase enzymes (FTases) expressed in different conditions. FTases were expressed in LB, minimal auto-induction or complex auto-induction at 20, 28 or 37° C. Soluble cell lysate was incubated with 625 mM sucrose in simulated intestinal fluid at 37° C. Activity was assessed by release of free glucose. Data is shown for 24 h expression in complex auto-induction medium at 28° C. Empty vector (EV) was used as control. Background signal from the buffer was subtracted. Note that the linear range of the assay is A340=0.015 to 1.206. *Samples were diluted 100-fold. Results discussed in Example 3.

FIG. 2. Expression and purification of FTases. (A) SDS-PAGE of eluted proteins. Bands at expected MW are indicted by arrows. Enzymes with low expression were loaded twice (a, b). (B) Expression yields of inulosucrases per L culture. The yields of proteins of SEQ ID NOs: 2 and 4 were not quantifiable (n.q.). *Yield of protein of SEQ ID NO: 7 was calculated from the observed band in (A).

FIG. 3. Activity of inulosucrases in simulated intestinal conditions. 10 μg/mL inulosucrase was incubated with 500 mM sucrose at 37° C. (A, B) Transfructosylation of inulosucrases was monitored without (A) and with (B) pancreatin. Fructose in FOS was calculated as the difference between free glucose and free fructose. (C, D) Hydrolytic activity was measured by monitoring free fructose in the presence (D) and absence (C) of pancreatin. Error bars represent+1 standard deviation. Some error bars are too small to be visible. N=3.

FIG. 4. Activity of FTases at low sucrose concentrations. SEQ ID NOs: 1 (FIG. 4A), 3 (FIG. 4B), 6 (FIG. 4C) and 8 (FIG. 4D) were incubated in simulated duodenal conditions at 37° C. and approximately pH 5.5 for 30 min with various concentrations of sucrose. FOS production was inferred from release of free glucose or fructose. Error bars represent±1 standard deviation. N=4, except SEQ ID NO 3 where N=3. Results are described in example 7.

FIG. 5. Enzyme concentration dependence of sucrose conversion. Serial dilutions of SEQ ID NO: 1 from 50 μg/mL to 5 μg/mL were incubated with 125 mM (4.2%) sucrose in simulated duodenal conditions for 30 min at 37° C. Conversion of available fructose into FOS was inferred from release of free glucose/fructose. Signal from simulated intestinal phase without FTase was subtracted. Data are from three separate experiments, each experiment n=5. Error bars represent±1 standard deviation. Results are described in example 8.

FIG. 6. Rate of conversion of sucrose to FOS. 10 μg/mL SEQ ID NO: 1 was incubated with 125 mM (4.2%) sucrose in simulated duodenal conditions for at 37° C. At each time point the reaction was stopped and conversion of available fructose into FOS was inferred from release of free glucose/fructose. Signal from simulated intestinal phase without FTase was subtracted. Data are from three separate experiments, each experiment n=5. Error bars represent±1 standard deviation. Results are described in example 9.

FIG. 7. Sucrose conversion from commercially available chocolate bar. Conversion of sucrose from 22.5 g serving of a Cadbury's™ Dairy Milk™ chocolate bar was tested in a dynamic gut model. The chocolate was passed through the gastric phase. After the gastric phase (t=60 min) bile acids were added, followed by 475 mg SEQ ID NO 1 or an equivalent volume of water. Pancreatic secretions were pumped into the digest for the next 120 min to a total volume of 95 mL. FOS production was inferred from released free glucose/fructose. Error bars represent+1 standard deviation. N=3. Results are described in example 10.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

In addition as used in this specification and the appended claims, the singular forms “a”, “an”, and “the” include plural referents unless the content clearly dictates otherwise. Thus, for example, reference to “a fructosyltransferase” includes two or more fructosyltransferases, reference to “a fructooligosaccharide” includes two or more such fructooligosaccharides and the like.

All publications, patents and patent applications cited herein, whether supra or infra, are hereby incorporated by reference in their entirety.

### Definitions

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “amino acid” in the context of the present disclosure is used in its broadest sense and is meant to include organic compounds containing amine (NH2) and carboxyl (COOH) functional groups, along with a side chain (e.g., a R group) specific to each amino acid. In some embodiments, the amino acids refer to naturally occurring L α-amino acids or residues. The commonly used one and three letter abbreviations for naturally occurring amino acids are used herein: A=Ala; C=Cys; D=Asp; E=Glu; F=Phe; G=Gly; H=His; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as β-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The terms “polypeptide”, and “peptide” are interchangeably used herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. Polypeptides can also undergo maturation or post-translational modification processes that may include, but are not limited to: glycosylation, proteolytic cleavage, lipidization, signal peptide cleavage, propeptide cleavage, phosphorylation, and such like. A peptide can be made using recombinant techniques, e.g., through the expression of a recombinant or synthetic polynucleotide. A recombinantly produced peptide it typically substantially free of culture medium, e.g., culture medium represents less than about 20%, more preferably less than about 10%, and most preferably less than about 5% of the volume of the protein preparation.

The term “protein” is used to describe a folded polypeptide having a secondary or tertiary structure. The protein may be composed of a single polypeptide, or may comprise multiple polypepties that are assembled to form a multimer. The multimer may be a homooligomer, or a heterooligmer. The protein may be a naturally occurring, or wild type protein, or a modified, or non-naturally, occurring protein. The protein may, for example, differ from a wild type protein by the addition, substitution or deletion of one or more amino acids.

A “variant” of a protein encompass peptides, oligopeptides, polypeptides, proteins and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

For all aspects and embodiments of the present invention, a “variant” typically has at least 50%, 60%, 70%, 80%, 90%, 95%, 96%, 97%, 98% or 99% complete sequence identity to the amino acid sequence of the corresponding wild-type protein. Sequence identity can also be to a fragment or portion of the full length polynucleotide or polypeptide. Hence, a sequence may have only 50% overall sequence identity with a full length reference sequence, but a sequence of a particular region, domain or subunit could share 80%, 90%, or as much as 99% sequence identity with the reference sequence.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence (e.g., substitutions, truncations, or insertions), post-translational modifications and/or functional properties (e.g., altered characteristics) when compared to the wild-type gene or gene product. It is noted that naturally occurring mutants can be isolated; these are identified by the fact that they have altered characteristics when compared to the wild-type gene or gene product. Methods for introducing or substituting naturally-occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. Methods for introducing or substituting non-naturally-occurring amino acids are also well known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. They may also be produced by naked ligation if the mutant monomer is produced using partial peptide synthesis. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, | Met | hydrophobic, neutral |
| | neutral | | |
| Cys | polar, hydrophobic, neutral | Asn | polar, hydrophilic, |
| | | | neutral |
| Asp | polar, hydrophilic, charged | Pro | hydrophobic, neutral |
| | (−) | | |
| Glu | polar, hydrophilic, charged | Gln | polar, hydrophilic, |
| | (−) | | neutral |
| Phe | aromatic, hydrophobic, | Arg | polar, hydrophilic, |
| | neutral | | charged (+) |
| Gly | aliphatic, neutral | Ser | polar, hydrophilic, |
| | | | neutral |
| His | aromatic, polar, hydrophilic, | Thr | polar, hydrophilic, |
| | charged (+) | | neutral |
| Ile | aliphatic, hydrophobic, | Val | aliphatic, hydrophobic, |
| | neutral | | neutral |
| Lys | polar, hydrophilic, | Trp | aromatic, hydrophobic, |
| | charged(+) | | neutral |
| Leu | aliphatic, hydrophobic, | Tyr | aromatic, polar, |
| | neutral | | hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

A mutant or modified protein, monomer or peptide can also be chemically modified in any way and at any site. A mutant or modified monomer or peptide may be chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant of modified protein, monomer or peptide may be chemically modified by the attachment of any molecule. For instance, the mutant of modified protein, monomer or peptide may be chemically modified by attachment of a dye or a fluorophore.

### Methods of Using Fructosyltransferases

In one aspect, the disclosure relates to an in vivo method of reducing fructose uptake in a subject. The method comprises administering to the subject an isolated fructosyltransferase. In another aspect, the disclosure relates to an in vivo method of reducing the formation of fructose via metabolism of sucrose in a subject, comprising administering to the subject an isolated fructosyltransferase. The method may be a therapeutic method or a non-therapeutic method as described in more detail herein. The method may be a non-therapeutic method which does not comprise treatment of the human or animal body by therapy or surgery. Also provided is a fructosyltransferase (e.g. an isolated fructosyltransferase) for use in reducing fructose uptake in a subject. Also provided is a fructosyltransferase (e.g. an isolated fructosyltransferase) for use in reducing the formation of fructose via metabolism of sucrose in a subject. Also provided is use of a fructosyltransferase (e.g. an isolated fructosyltransferase) in the manufacture of an agent for reducing fructose uptake in a subject. Also provided is use of a fructosyltransferase (e.g. an isolated fructosyltransferase) in the manufacture of an agent for reducing the formation of fructose via metabolism of sucrose in a subject.

As explained in more detail below, the administration of an isolated fructosyltransferase leads to the conversion of sucrose (typically present as a result of consumption of sugars in food) to fructooligosaccharides such as inulin and/or levan. In other words, fructosyltransferases are enzymes which catalyse this reaction. Fructosyltransferase enzymes for use in the disclosed methods are described in more detail herein.

Thus, in one aspect the method provided herein is a method of producing a fructooligosacharide in a subject in vivo, the method comprising administering to the subject an isolated fructosyltransferase and thereby converting sucrose to said fructooligosacharide. The fructooligosaccharides is typically inulin or levan, most usually inulin. The method may be a therapeutic method or a non-therapeutic method as described in more detail herein. The method may be a non-therapeutic method which does not comprise treatment of the human or animal body by therapy or surgery. Also provided is a fructosyltransferase (e.g. an isolated fructosyltransferase) for use in producing a fructooligosacharide in a subject in vivo. Also provided is use of a fructosyltransferase (e.g. an isolated fructosyltransferase) in the manufacture of an agent for producing a fructooligosacharide in a subject in vivo.

Inulin is a naturally occurring fructan-type oligosaccharide first reported in 1804 by Rose as a carbohydrate isolated from Inula helenum (Elecampane). Later that century (1879) inulin was mentioned in the “Pharmacographia: a history of the principle drugs of vegetable origin met within Great Britain and British India” by Friedrich Flueckinger and Daniel Hanbury. Inulin is naturally found in high concentrations in Jerusalem artichoke, chicory root, garlic, asparagus root and to a lesser degree in onion, leek, banana and wheat (Kaur & Gupta 2002). As such, the average daily intake of inulin ranges between 1 and 10 g in a typical Western diet, wherein European intake (3-11 g) is higher than American (1-4 g) (Coussement 1999).

Inulin is composed of repeating β-D-fructosyl units linked by glycosidic bonds, and a chain nucleating α-D-glucosyl group. Short inulin chains (less than 10 fructose units) are also referred to as oligofructose. Inulin is distinguished from the compositionally similar oligosaccharide known as levan in the nature of the glycosidic bonds in the polymers: in inulin, the bonds are (2-1), whereas in levan the bonds are (2→6). The relationship between these structures is shown below:

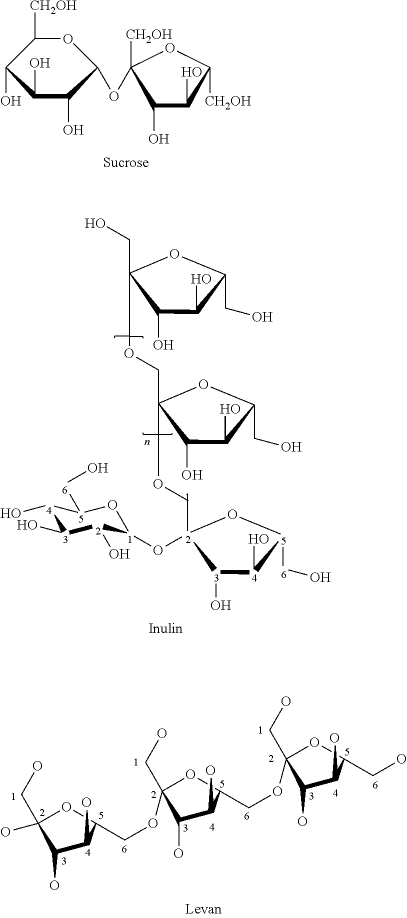

The methods provided herein are based at least in part on the recognition that fructooligosaccharides such as inulin and levan cannot be naturally metabolised by subjects such as mammals, e.g. humans. Accordingly, such fructooligosaccharides are commonly considered to be “calorie-free” dietary fibres. The production of fructooligosaccharides such as inulin and levan in vivo reduces the amount of sucrose available for metabolism into its component monomers (glucose and fructose) by enzymes in the body such as sucrases, isomaltase glycoside hydrolases, and invertases. In other words, by reducing the concentration of the sucrose substrate for these enzymes, the production of free glucose and especially free fructose is reduced. Because the concentration of free glucose and fructose in the body is decreased, the uptake of these molecules is reduced.

A further advantage of the disclosed methods is that the fructooligosaccharides such as inulin produced therein is associated with its own health benefits apart from those arising from the reduction of sucrose. Thus, a synergistic advantage arises from the administration of isolated fructosyltransferase, as dual benefits arise from both reducing sucrose levels and also increasing fructooligosaccharides (e.g. inulin) levels.

One primary driver of health benefits arising from fructooligosaccharides such as inulin is a shift in the colonic microbiome. Unlike many dietary fibres, fermentation of fructooligosaccharides such as inulin is selective. Such fructooligosaccharides can be metabolised by genera associated with gut health including lactobacilli, bifidobacteria and fusobacteria, leading to their proliferation. This proliferation reduces the proportion of pathogenic/opportunistic bacteria in the gut, such as certain strains of E. coli, Clostridia and Candida. Thus, the methods and uses provided herein may comprise administering an isolated fructosyltransferase to a subject in order to improve the subject's microbiome, e.g. by promoting the proliferation of bacteria such as lactobacilli, bifidobacteria and fusobacteria. Further benefits in terms of reduction of intrahepatocellular and intramyocellular lipids have been associated with fructooligosaccharides such as inulin.

The fructooligosaccharides generated in the methods provided herein, e.g. the inulin or levan, is typically at least 2, such as at least 3, e.g. at least 4, e.g. at least 5, e.g. at least 10, such as at least 20, e.g. at least 30, e.g. at least 40, e.g. at least 50, e.g. at least 100, monomer units in length. The fructooligosaccharides is often of from² to 200 monomer units in length, such as from 5 to 100 monomer units, such as 10 to 80 monomer units, e.g. from²⁰ to 60 monomer units, such as from 30 to 50 monomer units in length.

Typically, in the methods provided herein, the fructosyltransferase is administered to a subject in the form of a nutraceutical or pharmaceutical composition, or in the form of a food composition or foodstuff. Such compositions per se are also expressly provided herein. Nutraceutical and pharmaceutical compositions are described in more detail herein. Food compositions and foodstuffs are described in more detail herein.

The isolated fructosyltransferase, or the composition comprising such fructosyltransferase, is typically orally administered to the subject.

### Fructosyltranserases

The methods disclosed herein comprise the administration of isolated fructosyltransferase enzymes to a subject.

As those skilled in the art will appreciate, any suitable isolated fructosyltransferase can be used in the methods and products provided herein.

As used herein, the term isolated refers to the enzyme being extra cellular. The enzyme is typically purified from a cellular host. An isolated enzyme as used herein is not provided within a bacterial or fungal host. Administration of a bacteria or fungus comprising a fructosyltransferase enzyme to an organism does not correspond to administration of the isolated enzyme to the organism. The term “isolated enzyme” thus does not embrace whole cells such as bacterial or fungal cells.

Those skilled in the art will appreciate, however that the term “isolated enzyme” does not require that nothing apart from the enzyme is present. As explained in more detail herein, an “isolated enzyme” may be administered in the form of a nutraceutical or pharmaceutical composition. An “isolated enzyme” may be comprised in a food composition or foodstuff Impurities may also be present. However, often no impurities are present, e.g. the enzyme is substantially purified. Often a composition comprising an isolated enzyme as defined herein may be substantially free or free of impurities such as host DNA (e.g. DNA from an organism such as a bacteria or yeast in which the enzyme may be expressed). As explained below in more detail, a nutraceutical composition typically comprises the isolated enzyme and one or more excipients, diluents, or other nutraceutically acceptable additive(s). Similarly, pharmaceutical compositions typically comprise the isolated enzyme and one or more excipients, diluents, or other pharmaceutically acceptable additive(s). A food composition or foodstuff comprising an isolated enzyme as described herein typically comprises the isolated enzyme and one or more carbohydrates, fats, lipids, flavouring agent, colouring agent, etc.

The fructosyltransferase enzyme used in the disclosed methods is functional, i.e. it is capable of converting sucrose to fructooligosaccharides such as inulin and/or levan. A denatured enzyme is typically not functional. Thus, administration of a foodstuff to a subject wherein the foodstuff has been pretreated with an fructosyltransferase enzyme or an organism expressing a fructosyltransferase enzyme does not correspond to administration of an isolated fructosyltransferase to the subject. In such foodstuffs the enzyme with which the foodstuff has been pretreated is typically denatured or inactivated such that it is not functional, e.g. via heat treatment during cooking processes e.g. baking. This contrasts with embodiments of the present disclosure in which the enzyme is administered in a foodstuff such that it retains enzymatic activity in vivo, e.g. in the digestive system e.g. in the small intestine and/or the stomach.

The enzyme may be expressed intracellularly in the cellular host and isolated by being purified from the host. For example, an intracellular enzyme may be isolated via cell lysis followed by purification of the cell lysate.

Alternatively, a fructosyltransferase may be an extracellular enzyme, e.g. an enzyme that is expressed by the organism by secretion into the expression medium. Such enzymes may be isolated by purification of the expression medium without requiring cell lysis.

A fructosyltransferase may be expressed naturally in a cellular organism as an intracellular enzyme and be modified in order to be excreted from the cell as an extracellular enzyme. For example, a fructosyltransferase can be modified by deleting a cell wall anchor domain, e.g. at the C terminus of the protein sequence, in order to promote secretion into the expression medium. A fructosyltransferase can be modified by deleting a signal peptide if present from the protein sequence.

A fructosyltransferase may be expressed in any suitable organism. Protein expression is routine to those skilled in the art and is described in, for example, references such as Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016).

Typically, a fructosyltransferase for use in the disclosed methods is expressed in cells such as in bacterial cells, yeast cells, or insect cells. Bacterial cells are typically used. Many bacteria are recognised as being GRAS (Generally Recognised as Safe) and thus suitable for human consumption. Whilst the disclosed methods focus on the administration of isolated enzymes, the production of such enzymes from GRAS organisms is beneficial in conferring GRAS status on the isolated enzymes. Accordingly, in some embodiments the bacteria used to express the fructosyltransferase for use in the disclosed methods is certified as being GRAS. When a bacterium is used to express the fructosyltransferase enzyme for use in the disclosed products and methods, any suitable bacterium may be used. In some other embodiments the yeast used to express the fructosyltransferase for use in the disclosed methods is certified as being GRAS. When a yeast is used to express the fructosyltransferase enzyme for use in the disclosed products and methods, any suitable yeast may be used. For example, the fructosyltransferase is typically expressed by or is obtainable by expression from an organism of genus Escherichia, Lactobacillus, Saccharomyces, Bacillus, Pichia, Trichoderma or Aspergillus; preferably E. coli, S. cerevisiae, B. subtilis, P. pastoris, T reesei, A. niger, or A. oryzae. For example, the fructosyltransferase is typically expressed by or is obtainable by expression from an organism of genus Escherichia, Lactobacillus, Saccharomyces or Bacillus, such as Escherichia or Bacillus, preferably E. coli, S. cerevisiae or B. subtilis. In some embodiments the fructosyltransferase is expressed by or is obtainable by expression from an organism of genus Escherichia, Bacillus or Pichia, such as E. coli, B. subtilis or P. pastoris. Accordingly, in some embodiments the disclosed methods comprise expressing the fructosyltransferase enzyme in an organism of genus Escherichia, Lactobacillus, Saccharomyces, Bacillus, Pichia, Trichoderma or Aspergillus, such as Escherichia, Bacillus or Pichia, isolating the fructosyltransferase and then administering the isolated fructosyltransferase to a subject. In some embodiments the disclosed methods comprise expressing the fructosyltransferase enzyme in E. coli, S. cerevisiae, B. subtilis, P. pastoris, T reesei, A. niger, or A. oryzae, such as E. coli, B. subtilis or P. pastoris, isolating the fructosyltransferase and then administering the isolated fructosyltransferase to a subject. In some embodiments the disclosed methods comprise expressing the fructosyltransferase enzyme in an organism of genus Escherichia, Lactobacillus or Bacillus, such as Escherichia or Bacillus, preferably E. coli or B. subtilis, isolating the fructosyltransferase and then administering the isolated fructosyltransferase to a subject.

A fructosyltransferase for use in the disclosed methods may be isolated by cell lysis if required. Cell lysis can be conducted using any suitable method. For example, cells can be physical lysed, e.g. using a French press, or by sonication in a suitable buffer. Such buffers are commercially available e.g. from Qiagen.

Impure enzyme solutions can be purified for use in the disclosed methods by any suitable means. Typically fructosyltransferase enzymes can be purified using suitable chromatographic methods which are readily accessible to those skilled in the art. Suitable chromatographic methods include ion exchange chromatography (e.g. anion exchange or cation exchange chromatography), size exclusion chromatography, and/or hydrophobic interaction chromatography. Affinity chromatography may also be used. Any suitable affinity system can be used. For example, a fructosyltransferase enzyme may be tagged with a tag such as poly-histidine tag (e.g. HHHH, HHHHHH, or HHHHHHHH) and purified on a metal-containing column, e.g. a nickel or cobalt nitriloacetic acid column. Other purification tags include peptide tags such as Strep (WSHPQFEK), FLAG (DYKDDDDK), Human influenza hemagglutinin (HA) (YPYDVPDYA), Myc (EQKLISEED), and V5 (GKPIPNPLLGLDST), etc which may be purified using suitable columns. Purification tags may be cleavable or non-cleavable. The selection of suitable purification techniques is routine to those skilled in the art.

In yet another aspect, a fructosyltransferase for use in the methods provided herein can be expressed in a cell free expression system. For example, a fructosyltransferase enzyme can be expressed by in vitro transcription/translation (IVTT) from a suitable expression plasmid. Kits for conducting IVTT are commercially available from suppliers such as New England Biolabs (NEB).

The fructosyltransferase may be any suitable enzyme that is capable of converting sucrose to one or more fructooligosaccharides.

Accordingly, in some embodiments the provided method is a method of reducing fructose uptake in a subject and producing one or more fructooligosaccharides in vivo, comprising administering to the subject an isolated fructosyltransferase and thereby converting sucrose to one or more fructooligosaccharides in vivo. Such a method may be therapeutic or non-therapeutic as described herein. Such a method may be a non-therapeutic method which does not comprise treatment of the human or animal body by therapy or surgery. In some embodiments the method is a method of reducing the formation of fructose via metabolism of sucrose in a subject and producing one or more fructooligosaccharides in vivo, comprising administering to the subject an isolated fructosyltransferase and thereby converting sucrose to one or more fructooligosaccharides in vivo. Such methods may be therapeutic or non-therapeutic as described herein. Such a method may be a non-therapeutic method which does not comprise treatment of the human or animal body by therapy or surgery.

Also provided herein is a fructosyltransferase (e.g. an isolated fructosyltransferase) for use in reducing fructose uptake in a subject and producing one or more fructooligosaccharides in vivo. Said use may comprise administering to the subject an isolated fructosyltransferase and thereby converting sucrose to one or more fructooligosaccharides in vivo. Also provided is a fructosyltransferase (e.g. an isolated fructosyltransferase) for use in reducing the formation of fructose via metabolism of sucrose in a subject and producing one or more fructooligosaccharides in vivo. Said use may comprise administering to the subject an isolated fructosyltransferase and thereby converting sucrose to one or more fructooligosaccharides in vivo. Also provided is the use of a fructosyltransferase (e.g. an isolated fructosyltransferase) in the manufacture of an agent for reducing fructose uptake in a subject and producing one or more fructooligosaccharides in vivo. Also provided is the use of a fructosyltransferase (e.g. an isolated fructosyltransferase) in the manufacture of an agent for reducing the formation of fructose via metabolism of sucrose in a subject and producing one or more fructooligosaccharides in vivo.

The methods also typically involve reducing glucose production, although often at a lower level than the reduction of fructose production. For example, the initial monomer in inulin is typically glucose and thus free glucose levels are reduced by production of inulin.

Accordingly, in some embodiments the provided method is a method of reducing glucose uptake in a subject and producing one or more fructooligosaccharides in vivo, comprising administering to the subject an isolated fructosyltransferase and thereby converting sucrose to one or more fructooligosaccharides in vivo. In some embodiments the method is a method of reducing the formation of glucose via metabolism of sucrose in a subject and producing one or more fructooligosaccharides in vivo, comprising administering to the subject an isolated fructosyltransferase and thereby converting sucrose to one or more fructooligosaccharides in vivo. Such methods may be therapeutic or non-therapeutic as described herein. Such methods may be non-therapeutic methods which do not comprise treatment of the human or animal body by therapy or surgery.

Also provided herein is a fructosyltransferase (e.g. an isolated fructosyltransferase) for use in reducing glucose uptake in a subject and producing one or more fructooligosaccharides in vivo. Said use may comprise administering to the subject an isolated fructosyltransferase and thereby converting sucrose to one or more fructooligosaccharides in vivo. Also provided is a fructosyltransferase (e.g. an isolated fructosyltransferase) for use in reducing the formation of glucose via metabolism of sucrose in a subject and producing one or more fructooligosaccharides in vivo. Said use may comprise administering to the subject an isolated fructosyltransferase and thereby converting sucrose to one or more fructooligosaccharides in vivo. Also provided is the use of a fructosyltransferase (e.g. an isolated fructosyltransferase) in the manufacture of an agent for reducing glucose uptake in a subject and producing one or more fructooligosaccharides in vivo. Also provided is the use of a fructosyltransferase (e.g. an isolated fructosyltransferase) in the manufacture of an agent for reducing the formation of glucose via metabolism of sucrose in a subject and producing one or more fructooligosaccharides in vivo.

Most often, the fructosyltransferase is an inulosucrase or a levansucrase.

The fructosyltransferase may be an inulosucrase that is capable of converting sucrose to inulin. The fructosyltransferase may be an inulosucrase of EC class 2.4.1.9.

Accordingly, in some embodiments the provided method is a method of reducing fructose uptake in a subject and producing inulin in vivo, comprising administering to the subject an isolated inulosucrase and thereby converting sucrose to inulin in vivo. In some embodiments the method is a method of reducing the formation of fructose via metabolism of sucrose in a subject and producing inulin in vivo, comprising administering to the subject an isolated inulosucrase and thereby converting sucrose to inulin in vivo. In some embodiments the provided method is a method of reducing glucose uptake in a subject and producing inulin in vivo, comprising administering to the subject an isolated inulosucrase and thereby converting sucrose to inulin in vivo. In some embodiments the method is a method of reducing the formation of glucose via metabolism of sucrose in a subject and producing inulin in vivo, comprising administering to the subject an isolated inulosucrase and thereby converting sucrose to inulin in vivo. In some embodiments the provided method is a method of reducing fructose and glucose uptake in a subject and producing inulin in vivo, comprising administering to the subject an isolated inulosucrase and thereby converting sucrose to inulin in vivo. In some embodiments the method is a method of reducing the formation of fructose and glucose via metabolism of sucrose in a subject and producing inulin in vivo, comprising administering to the subject an isolated inulosucrase and thereby converting sucrose to inulin in vivo. Such methods may be therapeutic or non-therapeutic as described herein. Such methods may be non-therapeutic methods which do not comprise treatment of the human or animal body by therapy or surgery.

Also provided herein is an isolated inulosucrase for use in: (i) reducing fructose uptake in a subject and producing inulin in vivo, wherein said use comprises administering to the subject the isolated inulosucrase and thereby converting sucrose to inulin in vivo; (ii) reducing the formation of fructose via metabolism of sucrose in a subject and producing inulin in vivo, wherein said use comprises administering to the subject the isolated inulosucrase and thereby converting sucrose to inulin in vivo; (iii) reducing glucose uptake in a subject and producing inulin in vivo, wherein said use comprises administering to the subject the isolated inulosucrase and thereby converting sucrose to inulin in vivo; (iv) reducing the formation of glucose via metabolism of sucrose in a subject and producing inulin in vivo, wherein said use comprises administering to the subject the isolated inulosucrase and thereby converting sucrose to inulin in vivo; (v) reducing fructose and glucose uptake in a subject and producing inulin in vivo, wherein said use comprises administering to the subject the isolated inulosucrase and thereby converting sucrose to inulin in vivo; or (vi) reducing the formation of fructose and glucose via metabolism of sucrose in a subject and producing inulin in vivo, wherein said use comprises administering to the subject the isolated inulosucrase and thereby converting sucrose to inulin in vivo. Also provided is the use of an isolated inulosucrase in the manufacture of an agent for application according to any one of (i) to (vi) above.

In other embodiments the fructosyltransferase is a levansucrase that is capable of converting sucrose to levan. The fructosyltransferase may be a levansucrase of EC class 2.4.1.10.

Accordingly, in some embodiments the provided method is a method of reducing fructose uptake in a subject and producing levan in vivo, comprising administering to the subject an isolated levansucrase and thereby converting sucrose to levan in vivo. In some embodiments the method is a method of reducing the formation of fructose via metabolism of sucrose in a subject and producing levan in vivo, comprising administering to the subject an isolated levansucrase and thereby converting sucrose to levan in vivo. In some embodiments the provided method is a method of reducing glucose uptake in a subject and producing levan in vivo, comprising administering to the subject an isolated levansucrase and thereby converting sucrose to levan in vivo. In some embodiments the method is a method of reducing the formation of glucose via metabolism of sucrose in a subject and producing levan in vivo, comprising administering to the subject an isolated levansucrase and thereby converting sucrose to levan in vivo. In some embodiments the provided method is a method of reducing fructose and glucose uptake in a subject and producing levan in vivo, comprising administering to the subject an isolated levansucrase and thereby converting sucrose to levan in vivo. In some embodiments the method is a method of reducing the formation of fructose and glucose via metabolism of sucrose in a subject and producing levan in vivo, comprising administering to the subject an isolated levansucrase and thereby converting sucrose to levan in vivo. Such methods may be therapeutic or non-therapeutic as described herein. Such methods may be non-therapeutic methods which do not comprise treatment of the human or animal body by therapy or surgery.

Also provided herein is an isolated levansucrase for use in: (a) reducing fructose uptake in a subject and producing levan in vivo, wherein said use comprises administering to the subject the isolated levansucrase and thereby converting sucrose to levan in vivo; (b) reducing the formation of fructose via metabolism of sucrose in a subject and producing levan in vivo, wherein said use comprises administering to the subject the isolated levansucrase and thereby converting sucrose to levan in vivo; (c) reducing glucose uptake in a subject and producing levan in vivo, wherein said use comprises administering to the subject the isolated levansucrase and thereby converting sucrose to levan in vivo; (d) reducing the formation of glucose via metabolism of sucrose in a subject and producing levan in vivo, wherein said use comprises administering to the subject the isolated levansucrase and thereby converting sucrose to levan in vivo; (e) reducing fructose and glucose uptake in a subject and producing levan in vivo, wherein said use comprises administering to the subject the isolated levansucrase and thereby converting sucrose to levan in vivo; or (f) reducing the formation of fructose and glucose via metabolism of sucrose in a subject and producing levan in vivo, wherein said use comprises administering to the subject the isolated levansucrase and thereby converting sucrose to levan in vivo. Also provided is the use of an isolated levansucrase in the manufacture of an agent for application according to any one of (a) to (f) above.

Often, the fructosyltransferase has a high affinity for sucrose. Typically, the fructosyltransferase has a Michaelis constant (KM) for sucrose of less than 15.6 mM, such as less than 12 mM, e.g. less than 10 mM, such as less than 5 mM.

Typically, the fructosyltransferase is capable of converting sucrose to fructooligosaccharides such as inulin under low concentrations of sucrose. Low concentrations of sucrose are typically considered to favour sucrose hydrolysis (to fructose and glucose) over transfructosylation. However, typically a fructosyltransferase enzyme described herein is capable of maintaining a high ratio of transfructosylation compared to hydrolysis (i.e. a high T/H ratio) even under conditions of low sucrose concentration. For example, a fructosyltransferase described herein is typically capable of converting sucrose to fructooligosaccharides with a T/H ratio of at least 0.05, such as at least 0.1, e.g. at least such as at least 0.25 or at least 0.3 at a sucrose concentration of about 0.5% (e.g. about w/w or w/v). A fructosyltransferase described herein is typically capable of converting sucrose to fructooligosaccharides with a T/H ratio of at least 0.1, such as at least 0.2, e.g. at least 0.3, such as at least 0.4, e.g. at least 0.42 at a sucrose concentration of about 1% (e.g. about 1% w/w or w/v).

Typically the fructosyltransferase is active at body temperature (e.g. at about 37° C.) and at about pH 4 to about pH 9, such as from about pH 6 to about pH 8. Sometimes the activity of the fructosyltransferase at a pH of from about 1 to about 2 may be less than 50%, such as less than 40%, e.g. less than 30%, for example less than 20%, such as less than 10%, e.g. less than 5%, such as less than 4%, less than 3%, less than 2%, or less than 1% of the maximum activity of the fructosyltransferase at a pH of from about 4 to about pH 9. Sometimes the fructosyltransferase may not be active at a pH of about 1 to about 2, e.g. about 1.5. The human stomach typically has a pH of around 1.5, whereas the small intestines typically have a pH of approximately pH 6-8.

In some embodiments the fructosyltransferase is not denatured at a pH of about 1 to about 2, e.g. about 1.5. In some embodiments, although the fructosyltransferase may be substantially enzymatically inactive at a pH of about 1 to about 2 (e.g. may have an enzymatic activity at a pH of from about 1 to about 2 of less than 50% of the maximum activity of the fructosyltransferase at a pH of from about 4 to about pH 9, as defined above), the fructosyltransferase may retain activity if exposed to conditions of higher pH. Thus, in one example a fructosyltransferase may be administered to a subject where it is exposed to conditions of low pH e.g. in the stomach, and the fructosyltransferase may be substantially inactive in such environments; and the fructosyltransferase may be enzymatically active in regions of higher pH such as in the small intestine. In some embodiments the fructosyltransferase may not be denatured by low pH such as a pH of from about 1 to about 2 for a period of from about 0.1 hour to about 10 hours, such as from about 0.5 hours to about 5 hours e.g. from about 2 hours to about 4 hours.

In some embodiments the sequence of the fructosyltransferase is not known. However usually the sequence of the fructosyltransferase is known. The sequence of the fructosyltransferase can be determined using techniques routine in the art, including via gene sequencing, Edman degradation, etc.

Typically, the fructosyltransferase is or comprises a polypeptide according to any one of SEQ ID NOs: 1 to 10 or a functional variant thereof. As used herein, a functional variant is a variant comprising an amino acid sequence related to but different from that of the reference sequence (e.g. one of SEQ ID NOs: 1-10) and which retains the ability to catalyse the production of one or more fructooligosaccharides from sucrose.

A functional variant may be a functional fragment, derivative or variant of an enzyme or amino acid sequence described herein. As those skilled in the art will appreciate, fragments of amino acid sequences include deletion variants of such sequences wherein one or more, such as at least 1, 2, 5, 10, 20, 50, 100, 200 or 300 amino acids are deleted. Deletion may occur at the C-terminus or N-terminus of the native sequence or within the native sequence. Typically, deletion of one or more amino acids does not influence the residues immediately surrounding the active site of an enzyme. Derivatives of amino acid sequences include post-translationally modified sequences including sequences which are modified in vivo or ex vivo. Many different protein modifications are known to those skilled in the art and include modifications to introduce new functionalities to amino acid residues, modifications to protect reactive amino acid residues or modifications to couple amino acid residues to chemical moieties such as reactive functional groups on linkers.

Derivatives of amino acid sequences include addition variants of such sequences wherein one or more, such as at least 1, 2, 5, 10, 20, 50, 100, 200 or 300 amino acids are added or introduced into the native sequence. Addition may occur at the C-terminus or N-terminus of the native sequence or within the native sequence. Typically, addition of one or more amino acids does not influence the residues immediately surrounding the active site of an enzyme.

Variants of amino acid sequences include sequences wherein one or more amino acid such as at least 1, 2, 5, 10, 20, 50, 100, 200 or 300 amino acid residues in the native sequence are exchanged for one or more non-native residues. Such variants can thus comprise point mutations or can be more profound e.g. native chemical ligation can be used to splice non-native amino acid sequences into partial native sequences to produce variants of native enzymes. Variants of amino acid sequences include sequences carrying naturally occurring amino acids and/or unnatural amino acids. Variants, derivatives and functional fragments of the aforementioned amino acid sequences retain at least some of the activity/functionality of the native/wild-type sequence. Preferably, variants, derivatives and functional fragments of the aforementioned sequences have increased/improved activity/functionality when compared to the native/wild-type sequence.

A variant typically has at least 50%, 60%, 70%, 80%, 90%, 95%, 96%, 97%, 98% or 99% complete sequence identity to the amino acid sequence of the corresponding wild-type protein. The sequence identity is typically determined over at least 50%, 60%, 70%, 80%, 90%, 95%, 96%, 97%, 98% or 99% of the reference sequence. The sequence identity may be determined over the region of the sequence comprising the active site of the protein.

Accordingly, in one embodiment the fructosyltransferase is or comprises a polypeptide according to SEQ ID NO: 1 or a functional variant thereof; e.g. the fructosyltransferase may consist or comprise of a polypeptide having at least 70% homology or identity to SEQ ID NO: 1 over the entire sequence.

In another embodiment the fructosyltransferase is or comprises a polypeptide according to SEQ ID NO: 2 or a functional variant thereof; e.g. the fructosyltransferase may consist or comprise of a polypeptide having at least 70% homology or identity to SEQ ID NO: 2 over the entire sequence.

In another embodiment the fructosyltransferase is or comprises a polypeptide according to SEQ ID NO: 3 or a functional variant thereof; e.g. the fructosyltransferase may consist or comprise of a polypeptide having at least 70% homology or identity to SEQ ID NO: 3 over the entire sequence.

In another embodiment the fructosyltransferase is or comprises a polypeptide according to SEQ ID NO: 4 or a functional variant thereof; e.g. the fructosyltransferase may consist or comprise of a polypeptide having at least 70% homology or identity to SEQ ID NO: 4 over the entire sequence. In some embodiments a variant of a polypeptide according to SEQ ID NO: 4 is a polypeptide according to SEQ ID NO: 4a; wherein SEQ ID NO: 4a corresponds to residues 39 to 701 of SEQ ID NO: 4. In other words, SEQ ID NO: 4a omits residues 1 to 38 of SEQ ID NO: 4.

In another embodiment the fructosyltransferase is or comprises a polypeptide according to SEQ ID NO: 5 or a functional variant thereof; e.g. the fructosyltransferase may consist or comprise of a polypeptide having at least 70% homology or identity to SEQ ID NO: 5 over the entire sequence. In some embodiments a variant of a polypeptide according to SEQ ID NO: 5 is a polypeptide according to SEQ ID NO: 5a; wherein SEQ ID NO: 5a corresponds to residues 32 to 453 of SEQ ID NO: 5. In other words, SEQ ID NO: 5a omits residues 1 to 31 of SEQ ID NO: 5.

In another embodiment the fructosyltransferase is or comprises a polypeptide according to SEQ ID NO: 6 or a functional variant thereof; e.g. the fructosyltransferase may consist or comprise of a polypeptide having at least 70% homology or identity to SEQ ID NO: 6 over the entire sequence. In some embodiments a variant of a polypeptide according to SEQ ID NO: 6 is a polypeptide according to SEQ ID NO: 6a; wherein SEQ ID NO: 6a corresponds to residues 39 to 701 of SEQ ID NO: 6. In other words, SEQ ID NO: 6a omits residues 1 to 38 of SEQ ID NO: 6.

In another embodiment the fructosyltransferase is or comprises a polypeptide according to SEQ ID NO: 7 or a functional variant thereof; e.g. the fructosyltransferase may consist or comprise of a polypeptide having at least 70% homology or identity to SEQ ID NO: 7 over the entire sequence. In some embodiments a variant of a polypeptide according to SEQ ID NO: 7 is a polypeptide according to SEQ ID NO: 7a; wherein SEQ ID NO: 7a corresponds to residues 20 to 654 of SEQ ID NO: 7. In other words, SEQ ID NO: 7a omits residues 1 to 19 of SEQ ID NO: 7.

In another embodiment the fructosyltransferase is or comprises a polypeptide according to SEQ ID NO: 8 or a functional variant thereof; e.g. the fructosyltransferase may consist or comprise of a polypeptide having at least 70% homology or identity to SEQ ID NO: 8 over the entire sequence.

In another embodiment the fructosyltransferase is or comprises a polypeptide according to SEQ ID NO: 9 or a functional variant thereof; e.g. the fructosyltransferase may consist or comprise of a polypeptide having at least 70% homology or identity to SEQ ID NO: 9 over the entire sequence. In some embodiments a variant of a polypeptide according to SEQ ID NO: 9 is a polypeptide according to SEQ ID NO: 9a; wherein SEQ ID NO: 9a corresponds to residues 30 to 472 of SEQ ID NO: 9. In other words, SEQ ID NO: 9a omits residues 1 to 29 of SEQ ID NO: 9.

In another embodiment the fructosyltransferase is or comprises a polypeptide according to SEQ ID NO: 10 or a functional variant thereof; e.g. the fructosyltransferase may consist or comprise of a polypeptide having at least 70% homology or identity to SEQ ID NO: 10 over the entire sequence. In some embodiments a variant of a polypeptide according to SEQ ID NO: 10 is a polypeptide according to SEQ ID NO: 10a; wherein SEQ ID NO: 10a corresponds to residues 30 to 484 of SEQ ID NO: 10. In other words, SEQ ID NO: 10a omits residues 1 to 29 of SEQ ID NO: 10.

The active site of a fructosyltransferase enzyme can be determined by any suitable means. The active site may be determined by X-ray crystallography, e.g. in the presence of a substrate. The active site may be determined by in silico homology modelling based on the experimentally or theoretically determined structures of similar enzymes, such as related fructosyltransferases. The active site may be determined by genetic studies e.g. by mutating or deleting portions of the enzyme and correlating the changes made with the activity of the resulting variant. Residues associated with the active sites of the polypeptides corresponding to SEQ ID NOs: 1 to 10 are shown in grey/bold/bold&underlined in the sequence listing.

In some embodiments the fructosyltransferase has at least 70% homology or identity to SEQ ID NO: 1, wherein said homology or identity is assessed relative to the amino acid sequence from position 126 to 483 of SEQ ID NO: 1.

In some embodiments the fructosyltransferase has at least 70% homology or identity to SEQ ID NO: 1, wherein said homology or identity is assessed relative to some or all of positions 126, 128, 129, 131, 153, 156, 157, 158, 159, 160, 161, 162, 163, 194, 195, 196, 197, 198, 213, 215, 221, 223, 225, 277, 278, 279, 280, 281, 282, 283, 298, 378, 379, 380, 381, 382, 383, 397, 399, 401, 402, 457, 458, 459, 476, 479, 480, 481, 482 and 483 of SEQ ID NO: 1. These residue are shown in grey/bold/bold&underlined in SEQ ID NO: 1. Corresponding residues are shown in grey/bold/bold&underlined in SEQ ID NOs: 2, 3, 4 and 6. For avoidance of doubt, the corresponding residues in SEQ ID NO: 2 comprise positions 127, 129, 130, 132, 154, 157, 158, 159, 160, 161, 162, 163, 164, 195, 196, 197, 198, 199, 214, 216, 222, 224, 226, 278, 279, 280, 281, 282, 283, 284, 299, 379, 380, 381, 382, 383, 384, 398, 400, 402, 403, 458, 459, 460, 477, 480, 481, 482, 483 and 484. The corresponding residues in SEQ ID NO: 3 comprise positions 126, 128, 129, 131, 153, 156, 157, 158, 159, 160, 161, 162, 163, 194, 195, 196, 197, 198, 213, 215, 221, 223, 225, 277, 278, 279, 280, 281, 282, 283, 298, 378, 379, 380, 381, 382, 383, 397, 399, 401, 402, 457, 458, 459, 476, 479, 480, 481, 482 and 483. The corresponding residues in SEQ ID NO: 4 comprise positions 231, 233, 234, 236, 258, 261, 262, 263, 264, 265, 266, 267, 268, 300, 301, 302, 303, 304, 319, 321, 327, 329, 331, 381, 382, 383, 384, 385, 386, 387, 402, 482, 483, 484, 485, 486, 487, 501, 503, 505, 506, 561, 562, 563, 580, 584, 585, 586 and 587, The corresponding residues in SEQ ID NO: 6 comprise positions 231, 233, 234, 236, 258, 261, 262, 263, 264, 265, 266, 267, 268, 300, 301, 302, 303, 304, 319, 321, 327, 329, 331, 381, 382, 383, 384, 385, 386, 387, 402, 482, 483, 484, 485, 486, 487, 501, 503, 505, 506, 561, 562, 563, 580, 583, 584, 585, 586 and 587,

In some embodiments the fructosyltransferase has at least 70% homology or identity to SEQ ID NO: 1, wherein said homology or identity is assessed relative to some or all of positions 128, 129, 153, 158, 159, 160, 161, 162, 194, 196, 197, 213, 223, 277, 278, 281, 282, 298, 379, 381, 382, 399, 402, 457, 458 and 480 of SEQ ID NO: 1. These residue are shown in bold/bold&underlined in SEQ ID NO: 1. Corresponding residues are shown in bold/bold&underlined in SEQ ID NOs: 2, 3, 4 and 6.

In some embodiments the fructosyltransferase has at least 70% homology or identity to SEQ ID NO: 1, wherein said homology or identity is assessed relative to some or all of positions 128, 129, 153, 158, 159, 160, 162, 196, 197, 281, 282, 298, 379, 381, 399, 402, 457, 458 and 480 of SEQ ID NO: 1. These residue are shown in bold&underlined in SEQ ID NO: 1. Corresponding residues are shown in bold&underlined in SEQ ID NOs: 2, 3, 4 and 6.

Without being bound by theory, the inventors believe that some or all of positions 126, 128, 129, 131, 153, 156, 157, 158, 159, 160, 161, 162, 163, 194, 195, 196, 197, 198, 213, 215, 221, 223, 225, 277, 278, 279, 280, 281, 282, 283, 298, 378, 379, 380, 381, 382, 383, 397, 399, 401, 402, 457, 458, 459, 476, 479, 480, 481, 482 and 483 are comprised in the active site of the protein of SEQ ID NO: 1. For example, the fructosyltransferase may have at least 80%, at least 90%, at least 95%, at least 96%, at least 97%, at least 98% or at least 99% sequence identity with SEQ ID NO: 1, wherein sequence identity is determined relative to positions 126, 128, 129, 131, 153, 156, 157, 158, 159, 160, 161, 162, 163, 194, 195, 196, 197, 198, 213, 215, 221, 223, 225, 277, 278, 279, 280, 281, 282, 283, 298, 378, 379, 380, 381, 382, 383, 397, 399, 401, 402, 457, 458, 459, 476, 479, 480, 481, 482 and 483 of SEQ ID NO: 1; preferably to positions 128, 129, 153, 158, 159, 160, 161, 162, 194, 196, 197, 213, 223, 277, 278, 281, 282, 298, 379, 381, 382, 399, 402, 457, 458 and 480 of SEQ ID NO: 1; more preferably to positions 128, 129, 153, 158, 159, 160, 162, 196, 197, 281, 282, 298, 379, 381, 399, 402, 457, 458 and 480 of SEQ ID NO: 1. The fructosyltransferase may have 100% sequence identity to the sequence of SEQ ID NO: 1 wherein the identity of the sequence is assessed relative to positions 126, 128, 129, 131, 153, 156, 157, 158, 159, 160, 161, 162, 163, 194, 195, 196, 197, 198, 213, 215, 221, 223, 225, 277, 278, 279, 280, 281, 282, 283, 298, 378, 379, 380, 381, 382, 383, 397, 399, 401, 402, 457, 458, 459, 476, 479, 480, 481, 482 and 483 of SEQ ID NO: 1; preferably to positions 128, 129, 153, 158, 159, 160, 161, 162, 194, 196, 197, 213, 223, 277, 278, 281, 282, 298, 379, 381, 382, 399, 402, 457, 458 and 480 of SEQ ID NO: 1; more preferably to positions 128, 129, 153, 158, 159, 160, 162, 196, 197, 281, 282, 298, 379, 381, 399, 402, 457, 458 and 480 of SEQ ID NO: 1.

In some embodiments the fructosyltransferase has at least 70% homology or identity to SEQ ID NO: 5 or 5a, wherein said homology or identity is assessed relative to the amino acid sequence from position 47 to 387 of SEQ ID NO: 5.

In some embodiments the fructosyltransferase has at least 70% homology or identity to SEQ ID NO: 5 or 5a, wherein said homology or identity is assessed relative to some or all of positions 47, 49, 50, 52, 73, 75, 80, 81, 82, 83, 84, 85, 86, 117, 118, 119, 120, 121, 204, 205, 206, 208, 209, 210, 211, 228, 292, 293, 294, 295, 296, 311, 312, 313, 316, 325, 361, 362, 363, 374, 376, 377 and 387 of SEQ ID NO: 5. These residue are shown in grey/bold/bold&underlined in SEQ ID NO: 5.

In some embodiments the fructosyltransferase has at least 70% homology or identity to SEQ ID NO: 5 or 5a, wherein said homology or identity is assessed relative to some or all of positions 47, 49, 50, 73, 80, 81, 82, 83, 84, 85, 86, 117, 119, 120, 121, 204, 205, 210, 211, 228, 292, 293, 295, 296, 311, 313, 316, 361, 362, 374 and 377 of SEQ ID NO: 5. These residue are shown in bold/bold&underlined in SEQ ID NO: 5.

In some embodiments the fructosyltransferase has at least 70% homology or identity to SEQ ID NO: 5 or 5a, wherein said homology or identity is assessed relative to some or all of positions 49, 50, 73, 82, 83, 84, 85, 86, 119, 120, 210, 211, 293, 295 and 361 of SEQ ID NO: 5. These residue are shown in bold&underlined in SEQ ID NO: 5.

Without being bound by theory, the inventors believe that some or all of positions 47, 49, 50, 52, 73, 75, 80, 81, 82, 83, 84, 85, 86, 117, 118, 119, 120, 121, 204, 205, 206, 208, 209, 210, 211, 228, 292, 293, 294, 295, 296, 311, 312, 313, 316, 325, 361, 362, 363, 374, 376, 377 and 387 are comprised in the active site of the protein of SEQ ID NO: 5. For example, the fructosyltransferase may have at least 80%, at least 90%, at least 95%, at least 96%, at least 97%, at least 98% or at least 99% sequence identity with SEQ ID NO: 5 or 5a, wherein sequence identity is determined relative to positions 47, 49, 50, 52, 73, 75, 81, 82, 83, 84, 85, 86, 117, 118, 119, 120, 121, 204, 205, 206, 208, 209, 210, 211, 228, 292, 293, 294, 295, 296, 311, 312, 313, 316, 325, 361, 362, 363, 374, 376, 377 and 387; preferably to positions 47, 49, 50, 73, 80, 81, 82, 83, 84, 85, 86, 117, 119, 120, 121, 204, 205, 210, 211, 228, 292, 293, 295, 296, 311, 313, 316, 361, 362, 374 and 377; more preferably to positions 49, 50, 73, 82, 83, 84, 85, 86, 119, 120, 210, 211, 293, 295 and 361 of SEQ ID NO: 5. The fructosyltransferase may have 100% sequence identity to the sequence of SEQ ID NO: 5 or 5a wherein the identity of the sequence is assessed relative to positions 47, 49, 50, 52, 73, 75, 80, 81, 82, 83, 84, 85, 86, 117, 118, 119, 120, 121, 204, 205, 206, 208, 209, 210, 211, 228, 292, 293, 294, 295, 296, 311, 312, 313, 316, 325, 361, 362, 363, 374, 376, 377 and 387; preferably to positions 47, 49, 50, 73, 80, 81, 82, 83, 84, 86, 117, 119, 120, 121, 204, 205, 210, 211, 228, 292, 293, 295, 296, 311, 313, 316, 361, 362, 374 and 377; more preferably to positions 49, 50, 73, 82, 83, 84, 85, 86, 119, 120, 210, 211, 293, 295 and 361 of SEQ ID NO: 5.

In some embodiments the fructosyltransferase has at least 70% homology or identity to SEQ ID NO: 8, wherein said homology or identity is assessed relative to the amino acid sequence from position 54 to 389 of SEQ ID NO: 8.

In some embodiments the fructosyltransferase has at least 70% homology or identity to SEQ ID NO: 8, wherein said homology or identity is assessed relative to some or all of positions 54, 55, 56, 57, 58, 59, 60, 61, 73, 74, 75, 76, 77, 78, 79, 80, 81, 82, 113, 114, 115, 116, 117, 138, 140, 151, 187, 188, 263, 265, 266, 290, 299, 300, 301, 304, 306, 336, 337, 338, 339, 340, 341, 364, 365, 366, 367, 368, 369, 370, 371, 372, 373, 374, 375, 376, 386, 387, 388 and 389 of SEQ ID NO: 8. These residue are shown in grey/bold/bold&underlined in SEQ ID NO: 8. Corresponding residues are shown in grey/bold/bold&underlined in SEQ ID NOs: 7. For avoidance of doubt, the corresponding residues in SEQ ID NO: 7 comprise positions 38, 39, 40, 41, 42, 43, 44, 45, 57, 58, 59, 60, 61, 62, 63, 64, 65, 66, 97, 98, 99, 100, 101, 122, 124, 135, 171, 172, 271, 273, 274, 299, 308, 309, 310, 313, 315, 348, 349, 350, 351, 352, 353, 377, 378, 379, 380, 381, 382, 383, 384, 385, 386, 387, 388, 399, 400, 401 and 402.

In some embodiments the fructosyltransferase has at least 70% homology or identity to SEQ ID NO: 8, wherein said homology or identity is assessed relative to some or all of positions 54, 55, 56, 57, 58, 59, 73, 74, 75, 76, 77, 78, 81, 115, 116, 187, 188, 265, 266, 290, 304, 338, 339, 340, 366, 368, 370, 371, 372, 373, 374 and 389 of SEQ ID NO: 8. These residue are shown in bold/bold&underlined in SEQ ID NO: 8. Corresponding residues are shown in bold/bold&underlined in SEQ ID NOs: 7.

In some embodiments the fructosyltransferase has at least 70% homology or identity to SEQ ID NO: 8, wherein said homology or identity is assessed relative to some or all of positions 54, 55, 56, 57, 58, 59, 74, 75, 116, 265, 338, 339, 366, 370, 372 and 373 of SEQ ID NO: 8. These residue are shown in bold&underlined in SEQ ID NO: 8. Corresponding residues are shown in bold&underlined in SEQ ID NOs: 7.

Without being bound by theory, the inventors believe that some or all of positions 54, 55, 56, 57, 58, 59, 60, 61, 73, 74, 75, 76, 77, 78, 79, 80, 81, 82, 113, 114, 115, 116, 117, 138, 140, 151, 187, 188, 263, 265, 266, 290, 299, 300, 301, 304, 306, 336, 337, 338, 339, 340, 341, 364, 365, 366, 367, 368, 369, 370, 371, 372, 373, 374, 375, 376, 386, 387, 388 and 389 of SEQ ID NO: 8 are comprised in the active site of the protein of SEQ ID NO: 8. For example, the fructosyltransferase may have at least 80%, at least 90%, at least 95%, at least 96%, at least 97%, at least 98% or at least 99% sequence identity with SEQ ID NO: 8, wherein sequence identity is determined relative to positions 54, 55, 56, 57, 58, 59, 60, 61, 73, 74, 75, 76, 77, 78, 79, 80, 81, 82, 113, 114, 115, 116, 117, 138, 140, 151, 187, 188, 263, 265, 266, 290, 299, 300, 301, 304, 306, 336, 337, 338, 339, 340, 341, 364, 365, 366, 367, 368, 369, 370, 371, 372, 373, 374, 375, 376, 386, 387, 388 and 389; preferably to positions 54, 55, 56, 57, 58, 59, 73, 74, 75, 76, 77, 78, 81, 115, 116, 187, 188, 265, 266, 290, 304, 338, 339, 340, 366, 368, 370, 371, 372, 373, 374 and 389; more preferably to positions 54, 55, 56, 57, 58, 59, 74, 75, 116, 265, 338, 339, 366, 370, 372 and 373 of SEQ ID NO: 8. The fructosyltransferase may have 100% sequence identity to the sequence of SEQ ID NO: 8 wherein the identity of the sequence is assessed relative to positions 54, 55, 56, 57, 58, 59, 60, 61, 73, 74, 75, 76, 77, 78, 79, 80, 81, 82, 113, 114, 115, 116, 117, 138, 140, 151, 187, 188, 263, 265, 266, 290, 299, 300, 301, 304, 306, 336, 337, 338, 339, 340, 341, 364, 365, 366, 367, 368, 369, 370, 371, 372, 373, 374, 375, 376, 386, 387, 388 and 389; preferably to positions 54, 55, 56, 57, 58, 59, 73, 74, 75, 76, 77, 78, 81, 115, 116, 187, 188, 265, 266, 290, 304, 338, 339, 340, 366, 368, 370, 371, 372, 373, 374 and 389; more preferably to positions 54, 55, 56, 57, 58, 59, 74, 75, 116, 265, 338, 339, 366, 370, 372 and 373 of SEQ ID NO: 8.

In some embodiments the fructosyltransferase has at least 70% homology or identity to SEQ ID NO: 9 or 9a, wherein said homology or identity is assessed relative to the amino acid sequence from position 54 to 406 of SEQ ID NO: 9.

In some embodiments the fructosyltransferase has at least 70% homology or identity to SEQ ID NO: 9 or 9a, wherein said homology or identity is assessed relative to some or all of positions 54, 56, 57, 59, 80, 83, 84, 85, 86, 87, 88, 89, 90, 132, 133, 134, 135, 136, 150, 151, 153, 161, 208, 213, 214, 215, 216, 217, 218, 219, 233, 311, 312, 313, 314, 315, 329, 331, 333, 334, 382, 383, 384, 400, 403, 404, 405 and 406 of SEQ ID NO: 9. These residue are shown in grey/bold/bold&underlined in SEQ ID NO: 9. Corresponding residues are shown in grey/bold/bold&underlined in SEQ ID NO: 10. For avoidance of doubt, the corresponding residues in SEQ ID NO: 10 comprise positions 63, 65, 66, 68, 89, 92, 93, 94, 95, 96, 97, 98, 99, 141, 142, 143, 144, 145, 159, 160, 162, 170, 218, 223, 224, 225, 226, 227, 228, 229, 243, 321, 322, 323, 324, 325, 339, 341, 343, 344, 392, 393, 394, 410, 413, 414, 415 and 416.

In some embodiments the fructosyltransferase has at least 70% homology or identity to SEQ ID NO: 9 or 9a, wherein said homology or identity is assessed relative to some or all of positions 56, 57, 80, 84, 85, 86, 87, 88, 89, 90, 132, 134, 135, 151, 213, 217, 218, 233, 311, 313, 314, 331, 334, 382, 383, 400, 404 and 406 of SEQ ID NO: 9. These residue are shown in bold/bold&underlined in SEQ ID NO: 9.

In some embodiments the fructosyltransferase has at least 70% homology or identity to SEQ ID NO: 9 or 9a, wherein said homology or identity is assessed relative to some or all of positions 56, 57, 86, 87, 88, 134, 135, 217, 218, 233, 311, 313, 331, 334, 382 and 404 of SEQ ID NO: 9. These residue are shown in bold&underlined in SEQ ID NO: 9.

Without being bound by theory, the inventors believe that some or all of positions 54, 56, 57, 59, 80, 83, 84, 85, 86, 87, 88, 89, 90, 132, 133, 134, 135, 136, 150, 151, 153, 161, 208, 213, 214, 215, 216, 217, 218, 219, 233, 311, 312, 313, 314, 315, 329, 331, 333, 334, 382, 383, 384, 400, 403, 404, 405 and 406 are comprised in the active site of the protein of SEQ ID NO: 9. For example, the fructosyltransferase may have at least 80%, at least 90%, at least 95%, at least 96%, at least 97%, at least 98% or at least 99% sequence identity with SEQ ID NO: 9 or 9a, wherein sequence identity is determined relative to positions 54, 56, 57, 59, 80, 83, 84, 85, 86, 87, 88, 89, 90, 132, 133, 134, 135, 136, 150, 151, 153, 161, 208, 213, 214, 215, 216, 217, 218, 219, 233, 311, 312, 313, 314, 315, 329, 331, 333, 334, 382, 383, 384, 400, 403, 404, 405 and 406; preferably to positions 56, 57, 84, 85, 86, 87, 88, 89, 90, 132, 134, 135, 151, 213, 217, 218, 233, 311, 313, 314, 331, 334, 382, 383, 400, 404 and 406; more preferably to positions 56, 57, 86, 87, 88, 134, 135, 217, 218, 233, 311, 313, 331, 334, 382 and 404 of SEQ ID NO: 9. The fructosyltransferase may have 100% sequence identity to the sequence of SEQ ID NO: 9 or 9a wherein the identity of the sequence is assessed relative to positions 54, 56, 57, 59, 80, 83, 84, 85, 86, 87, 88, 89, 90, 132, 133, 134, 135, 136, 150, 151, 153, 161, 208, 213, 214, 215, 216, 217, 218, 219, 233, 311, 312, 313, 314, 315, 329, 331, 333, 334, 382, 383, 384, 400, 403, 404, 405 and 406; preferably to positions 56, 57, 80, 84, 85, 86, 87, 88, 89, 90, 132, 134, 135, 151, 213, 217, 218, 233, 311, 313, 314, 331, 334, 382, 383, 400, 404 and 406; more preferably to positions 56, 57, 86, 87, 88, 134, 135, 217, 218, 233, 311, 313, 331, 334, 382 and 404 of SEQ ID NO: 9.

Sequence homology or identity can be determined as described above, e.g. based on sequence alignment of the sequence at issue with a reference sequence (e.g. SEQ ID NO: 1, 5, 5a, 8, 9 or 9a).

Thus, in some embodiments the fructosyltransferase has at least 70%, 80%, 90%, 95%, 96%, 97%, 98%, 99% or 100% homology or identity to any one of SEQ ID NOs: 1, 2, 3, 4, 4a, 5, 5a, 6, 6a, 7, 7a, 8, 9, 9a, 10, or 10a, wherein said homology or identity is assessed relative to the positions marked in grey/bold/bold&underlined in the relevant sequence. In some embodiments the fructosyltransferase has at least 70%, 80%, 90%, 95%, 96%, 97%, 98%, 99% or 100% homology or identity to any one of SEQ ID NOs: 1, 2, 3, 4, 4a, 5, 5a, 6, 6a, 7, 7a, 8, 9, 9a, 10, or 10a, wherein said homology or identity is assessed relative to the positions marked in bold/bold&underlined in the relevant sequence. In some embodiments the fructosyltransferase has at least 70%, 80%, 90%, 95%, 96%, 97%, 98%, 99% or 100% homology or identity to any one of SEQ ID NOs: 1, 2, 3, 4, 4a, 5, 6, 6a, 7, 7a, 8, 9, 9a, 10, or 10a, wherein said homology or identity is assessed relative to the positions marked in bold&underlined in the relevant sequence.6

Typically, in one embodiment the fructosyltransferase comprises alanine at the position corresponding to A182 of SEQ ID NO: 1. For example, in SEQ ID NO: 2 the position corresponding to A182 of SEQ ID NO 1 is A183. In SEQ ID NO: 3 the position corresponding to A182 of SEQ ID NO 1 is A182. In SEQ ID NO: 4 the position corresponding to A182 of SEQ ID NO 1 is V287. In SEQ ID NO: 6 the position corresponding to A182 of SEQ ID NO 1 is A287.

Typically, in another embodiment, the fructosyltransferase comprises:
phenylalanine at the position corresponding to F372 of SEQ ID NO: 8 and/or
glycine at the position corresponding to G373 of SEQ ID NO: 8; and/or
asparagine at the position corresponding to N77 of SEQ ID NO: 8 and/or
glycine at the position corresponding to G340 of SEQ ID NO: 8 and/or
glutamate at the position corresponding to E371 of SEQ ID NO: 8 and/or
alanine at the position corresponding to A374 of SEQ ID NO: 8; and/or
threonine at the position corresponding to T79 of SEQ ID NO: 8 and/or
serine at the position corresponding to S82 of SEQ ID NO: 8 and/or
serine at the position corresponding to 5299 of SEQ ID NO: 8 and/or
threonine at the position corresponding to T301 of SEQ ID NO: 8 and/or
alanine at the position corresponding to A336 of SEQ ID NO: 8 and/or
tryptophan at the position corresponding to W364 of SEQ ID NO: 8.

For example, in SEQ ID NO: 7 the position corresponding to F372 of SEQ ID NO: 8 is Y385; the position corresponding to G373 of SEQ ID NO: 8 is E386; the position corresponding to N77 of SEQ ID NO: 8 is D61; the position corresponding to G340 of SEQ ID NO: 8 is A352; the position corresponding to E371 of SEQ ID NO: 8 is Q384; the position corresponding to A374 of SEQ ID NO: 8 is Q387; the position corresponding to T79 of SEQ ID NO: 8 is D63; the position corresponding to S82 of SEQ ID NO: 8 is A66; the position corresponding to S299 of SEQ ID NO: 8 is Q308; the position corresponding to T301 of SEQ ID NO: 8 is S310; the position corresponding to A336 of SEQ ID NO: 8 is S348; and the position corresponding to W364 of SEQ ID NO: 8 is F377.

More preferably the fructosyltransferase comprises:
phenylalanine at the position corresponding to F372 of SEQ ID NO: 8 and/or
glycine at the position corresponding to G373 of SEQ ID NO: 8; and/or
asparagine at the position corresponding to N77 of SEQ ID NO: 8 and/or
glycine at the position corresponding to G340 of SEQ ID NO: 8 and/or
glutamate at the position corresponding to E371 of SEQ ID NO: 8 and/or
alanine at the position corresponding to A374 of SEQ ID NO: 8.

Still more preferably the fructosyltransferase comprises phenylalanine at the position corresponding to F372 of SEQ ID NO: 8 and/or glycine at the position corresponding to G373 of SEQ ID NO: 8.

Typically, the fructosyltransferase is soluble in aqueous solution. Solubility can be expressed as a GRAVY (Grand Average of Hydropathy) score which can be determined based on the amino acid sequence of the fructosyltransferase. Calculation of GRAVY scores is routine for those skilled in the art. The GRAVY value is typically calculated by adding the hydropathy value for each residue (Kyte and Doolittle; J Mol Biol 1982 157(1):105-32) and dividing by the length of the sequence. GRAVY scores can be easily determined using freely available software e.g. at https://www.bioinformatics.org/sms2/protein_gravy.html. Typically, the fructosyltransferase for use in the products and methods provided herein has a solubility GRAVY score of −0.4 or more negative than −0.4, such as at most −0.5, e.g. at most −0.6. GRAVY scores for some exemplary fructosyltransferase enzymes are provided in the examples. Typically the fructosyltransferase is an inulosucrase having a GRAVY score of −0.4 or more negative than −0.4.

Typically, the fructosyltransferase for use in the products and methods provided herein is derived from an organism of genus Lactobacillus, Bacillus, Leuconostoc, Streptomyces, Aspergillus, or Clostridium. More typically, the fructosyltransferase is derived from an organism of species Lactobacillus gasseri, Lactobacillus johnsonii, Lactobacillus reuteri, Bacillus agaradhaerens, Bacillus amyloliquefaciens, Bacillus megaterium, Bacillus subtilis, Leuconostoc citreum, Leuconostoc mesenteroides, Streptomyces viridochromogenes, Aspergillus acelatus, Aspergillus sydowii, or Clostridium acetobutylicum.

Those skilled in the art will appreciate that references to a protein being derived from a given organism refers to the original host organism that natively expresses the protein at issue. References to a protein being “derived” from a specific organism does not mean that the protein is necessarily expressed in practice in such an organism. For example, expression organisms such as E. coli transformed with appropriate expression vectors are often used to express proteins natively produced by other organisms. Practitioners are referred to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for further discussion of the use of non-native expression systems in order to produce proteins. The source organism for the fructosyltransferase may be chosen based on desired characteristics of the sequence. Desired characteristics include activity of the fructosyltransferase, its stability in storage, its resistance to proteases, etc. Protease resistance can be determined as described in the examples.

Thus, for example, the fructosyltransferase may be derived from an organism of genus Lactobacillus, Bacillus, Leuconostoc, Streptomyces, Aspergillus, or Clostridium; and may be expressed in an organism such as Escherichia, Lactobacillus, Saccharomyces, Bacillus, Pichia, Trichoderma or Aspergillus; preferably E. coli, S. cerevisiae, B. subtilis, P. pastoris, T reesei, A. niger, or A. oryzae. The fructosyltransferase may be derived from an organism of species Lactobacillus gasseri, Lactobacillus johnsonii, Lactobacillus reuteri, Bacillus agaradhaerens, Bacillus amyloliquefaciens, Bacillus megaterium, Bacillus subtilis, Leuconostoc citreum, Leuconostoc mesenteroides, Streptomyces viridochromogenes, Aspergillus acelatus, Aspergillus sydowii, or Clostridium acetobutylicum; and may be expressed in an organism such as Escherichia, Lactobacillus, Saccharomyces, Bacillus, Pichia, Trichoderma or Aspergillus; preferably E. coli, S. cerevisiae, B. subtilis, P. pastoris, T reesei, A. niger, or A. oryzae. The fructosyltransferase may be derived from an organism of genus Lactobacillus, Bacillus, Leuconostoc, Streptomyces, Aspergillus, or Clostridium; and may be expressed in an organism such as a bacterium of genus Escherichia or Bacillus or a yeast of genus Saccharomyces. The fructosyltransferase may be derived from an organism of species Lactobacillus gasseri, Lactobacillus johnsonii, Lactobacillus reuteri, Bacillus agaradhaerens, Bacillus amyloliquefaciens, Bacillus megaterium, Bacillus subtilis, Leuconostoc citreum, Leuconostoc mesenteroides, Streptomyces viridochromogenes, Aspergillus acelatus, Aspergillus sydowii, or Clostridium acetobutylicum; and may be expressed in E. coli, B. subtilis or S. cerevisiae. The fructosyltransferase may be derived from an organism of genus Lactobacillus, Bacillus, Leuconostoc, Streptomyces, Aspergillus, or Clostridium; and may be expressed in any suitable GRAS organism such as a GRAS bacterium, yeast or fungus, such as a GRAS bacterium or yeast.

### Nutraceutical Compositions

As mentioned above, in one embodiment of the methods provided herein, the fructosyltransferase is administered to a subject in the form of a nutraceutical composition. Such compositions per se are also expressly provided herein.

A nutraceutical composition as used herein typically comprises a fructosyltransferase, e.g. a fructosyltransferase as described herein, and one or more nutraceutically acceptable filler, stabilizing agent, colouring agent or flavouring agent.

Suitable excipients for use in the nutraceutical composition include:
fillers such as lactose, sucrose, magnesium stearate, glucose, plant cellulose, calcium carbonate etc;
stabilizers such as vitamin A, C, E, selenium, amino acids, methyl paraben, and propyl paraben;
anti-adherents;
binders such as lactose, sucrose, microcrystalline cellulose, malitol, sorbitol, xylitol, starches, arabic gums, gelatin, methylcellulose, carboxymethylcellulose or polyvinyl pyrrolidone;
diluents, e.g. lactose, dextrose, saccharose, cellulose, corn starch or potato starch;
disintegrants such as starch, alginic acid, alginates or sodium starch glycolate;
lubricants such as silica, talc, stearic acid, magnesium or calcium stearate and/or polyethylene glycols;
dyestuffs and other colouring agents such as FD&C Blue No. 1 (brilliant blue FCF), FD&C Blue No. 2 (indigotine), FD&C Green No. 3 (fast green FCF), FD&C Red No. 40 (allura red AC), FD&C Red No. 3 (erythrosine), FD&C Yellow No. 5 (tartrazine), and FD&C Yellow No. 6 (sunset yellow);
flavouring agents such as sweet almond oil, benzaldehyde, DL-menthol, ethyl acetate, ethyl vanillin, L-menthol, methyl salicylate, peppermint oil, peppermint spirit, and vanillin;
effervescing mixtures;
sweeteners; and
wetting agents, such as lecithin, polysorbates, and laurylsulphates;
   Any suitable combination of any of the aforementioned excipients can be used in the nutraceutical compositions provided and described in more detail herein. Such nutraceutical preparations may be manufactured in a known manner, for example, by means of mixing, granulating, tableting, sugar coating, or film coating processes.

Typically, a nutraceutical composition as described herein is formulated as a tablet, a troche, a lozenge, an aqueous or oily suspension, a dispersible powder or as granules. A powder may be obtained by e.g. lyophilisation.

Liquid dispersions for oral administration may be syrups, emulsions and suspensions. The syrups may contain as carriers, for example, saccharose or saccharose with glycerine and/or mannitol and/or sorbitol. Suspensions and emulsions may contain a carrier, for example a natural gum, agar, sodium alginate, pectin, methylcellulose, carboxymethylcellulose, or polyvinyl alcohol. Syrups may be formulated to avoid the use of sucrose.

Choice of formulation of the nutraceutical composition is within the capability of those skilled in the art, and may depend on factors such as cultural, societal or commercial preferences, the end consumer of the product, any specific foodstuff targeted, etc.

Typically, a nutraceutical composition as described herein is suitable for oral administration to the subject. Thus, the methods disclosed herein which comprise the use of a nutraceutical composition as described herein typically comprise orally administering the nutraceutical composition to the subject.

Typically, the nutraceutical composition is intended to release the active agent (i.e. the fructosyltransferase) in an appropriate part of the body, where it can be active in converting sucrose. For example, the nutraceutical composition may release the active fructosyltransferase in the small gastrointestinal tract, e.g. in the small intestine. Accordingly, a nutraceutical composition as described herein may comprise an enteric coating. Any suitable enteric coating material known in the art can be used. Suitable materials include but are not limited to methyl acrylate-methacrylic acid copolymers; cellulose acetate phthalate (CAP); cellulose acetate succinate; hydroxypropyl methyl cellulose (HMPC) and hydroxypropyl methyl cellulose phthalate; hydroxypropyl methyl cellulose acetate succinate (hypromellose acetate succinate; EIMPAS); polyvinyl acetate phthalate (PVAP); methyl methacrylate-methacrylic acid copolymers; shellac; cellulose acetate trimellitate; sodium alginate; zein and the like.

Typically, the nutraceutical composition is provided as a dietary supplement. The composition may be provided as a kit together with instructions for use. The composition may be provided in the form of a supplement to be taken before, with, or after consuming food.

Typically, the nutraceutical composition comprises only ingredients which are generally recognised as safe (GRAS). The components of the composition are typically food grade components.

The fructosyltransferase is typically stable in the nutraceutical composition under appropriate storage conditions for extended periods of time. For example, the fructosyltransferase may be stable for in excess of 1 day, 1 month, 1 year, etc, when stored under appropriate conditions. The necessary stability of the fructosyltransferase can be determined based on its application and the form of the composition in which it is provided and can be controlled using methods known in the art, including the use of high purity reagents and storage under appropriate conditions.

Typically, a nutraceutical composition will contain up to 85 wt % of the fructosyltransferase described herein. It may contain up to 50 wt %, up to 40 wt %, up to 30 wt %, up to 20 wt % or up to 10 wt % of the fructosyltransferase.

Typically, a nutraceutical composition may contain sufficient fructosyltransferase to produce from about 1 to about 100 g, such as from about 2 g to about 50 g, e.g. from about 5 g to about 20 g such as about 10 g of fructooligosaccharides within about 0.5 to 5 hours, such as within about 1 to about 3 hours, e.g. within about 2 hours under physiological conditions. A nutraceutical composition thus may comprise from about 1 to about 1000 mg of fructosyltransferase, such as from about 10 to about 100 mg e.g. about mg of fructosyltransferase per unit dose.

A nutraceutical composition may comprise from about 1 mg to about 100 mg such as from about 2 mg to about 50 mg e.g. from about 5 mg to about 20 mg such as from about 7 mg to about 15 mg, e.g. about 10 mg of fructosyltransferase per unit dose.

A nutraceutical composition may be capable of acting on from about 1% to about 100% (e.g. % w/w or % w/v) e.g. from about 1% to about 80%, such as from about 5% to about 50%, e.g. from about 10% to about 40%, e.g. from about 20 to about 30% of sucrose molecules available within about 1 minute to about 1 hour, e.g. within about 10 minutes to about 45 minutes, such as within about 15 minutes to about 30 minutes under physiological conditions.

Those skilled in the art will appreciate that “available fructose” represents the fructose present in sucrose to be converted. As explained herein, each sucrose molecule comprises one glucose unit and one fructose unit such that conversion of 100% available fructose monomer units corresponds to incorporation of 50% monomer units of sucrose. Thus, to a first approximation (discounting the terminal glucose unit on the fructooligosaccharide generated by the fructosyltransferase), conversion of 100% of all sucrose molecules present in a sample thus corresponds to 50% conversion of saccharide units in sucrose (100% conversion/incorporation of fructose units).

A nutraceutical composition may thus be capable of converting/incorporating from about 1% to about 100% e.g. from about 1% to about 80%, such as from about 5% to about 50%, e.g. from about 10% to about 40%, e.g. from about 20 to about 30% of available fructose into fructooligosaccharides within about 1 minute to about 1 hour, e.g. within about 10 minutes to about 45 minutes, such as within about 15 minutes to about 30 minutes under physiological conditions. In other words a nutraceutical composition as provided herein may be capable of converting/incorporating from about 1% to about 50% e.g. from about 1% to about 40%, such as from about 5% to about 30%, e.g. from about 10% to about 20%, of the saccharide units present in available sucrose into fructooligosaccharides within about 1 minute to about 1 hour, e.g. within about 10 minutes to about 45 minutes, such as within about 15 minutes to about 30 minutes under physiological conditions.

A nutraceutical composition may be administered to a subject at any suitable administration frequency. For example, a nutraceutical composition may be administered at least once per day, such as between about 1 and about 20 times a day, e.g. between about 1 and about 10 times a day, such as between 2 and 5 times a day, e.g. about 3 or 4 times a day.

Typically, a neutraceutical composition is used in non-therapeutic methods. Accordingly, provided herein is use of a neutraceutical composition as described herein in a method (e.g. a non-therapeutic method) of reducing fructose uptake; reducing formation of fructose via metabolism of sucrose; reducing glucose uptake and/or reducing formation of glucose via metabolism of sucrose; producing a fructooligosaccharide; suppressing appetite; and/or increasing satiety in a subject. Also provided is a method (e.g. a non-therapeutic method) of reducing fructose uptake; reducing formation of fructose via metabolism of sucrose; reducing glucose uptake and/or reducing formation of glucose via metabolism of sucrose; producing a fructooligosaccharide; suppressing appetite; and/or increasing satiety in a subject, comprising administering a nutraceutical composition as described herein to the subject. Further provided is a neutraceutical composition as described herein for use in a method (e.g. a non-therapeutic method) of reducing fructose uptake; reducing formation of fructose via metabolism of sucrose; reducing glucose uptake and/or reducing formation of glucose via metabolism of sucrose; producing a fructooligosaccharide; suppressing appetite; and/or increasing satiety in a subject. Still further provided is use of an isolated fructosyltransferase as described herein in the manufacture of a nutraceutical composition as described herein for the (typically non-therapeutic) reduction of fructose uptake; reduction of formation of fructose via metabolism of sucrose; reduction of glucose uptake and/or reduction of formation of glucose via metabolism of sucrose; production of a fructooligosaccharide; suppression of appetite; and/or increase in satiety in a subject. Such methods and uses are described in more detail herein.

### Pharmaceutical Compositions

In another embodiment of the methods provided herein, the fructosyltransferase is administered to a subject in the form of a pharmaceutical composition. Such compositions per se are also expressly provided herein.

A pharmaceutical composition as used herein typically comprises a fructosyltransferase, e.g. a fructosyltransferase as described herein, and one or more pharmaceutical acceptable carrier, excipient or diluent.

Suitable components for such use in the pharmaceutical composition include:
fillers such as lactose, sucrose, magnesium stearate, glucose, plant cellulose, calcium carbonate etc;
stabilizers such as vitamin A, C, E, selenium, amino acids, methyl paraben, and propyl paraben;
anti-adherents;
binders such as lactose, sucrose, microcrystalline cellulose, malitol, sorbitol, xylitol, starches, arabic gums, gelatin, methylcellulose, carboxymethylcellulose or polyvinyl pyrrolidone;
diluents, e.g. lactose, dextrose, saccharose, cellulose, corn starch or potato starch;
disintegrants such as starch, alginic acid, alginates or sodium starch glycolate;
lubricants such as silica, talc, stearic acid, magnesium or calcium stearate and/or polyethylene glycols;
dyestuffs and other colouring agents such as FD&C Blue No. 1 (brilliant blue FCF), FD&C Blue No. 2 (indigotine), FD&C Green No. 3 (fast green FCF),
FD&C Red No. 40 (allura red AC), FD&C Red No. 3 (erythrosine), FD&C Yellow No. 5 (tartrazine), and FD&C Yellow No. 6 (sunset yellow);
flavouring agents such as sweet almond oil, benzaldehyde, DL-menthol, ethyl acetate, ethyl vanillin, L-menthol, methyl salicylate, peppermint oil, peppermint spirit, and vanillin;
effervescing mixtures;
sweeteners; and
wetting agents, such as lecithin, polysorbates, and laurylsulphates;
   Any suitable combination of any of the aforementioned excipients can be used in the pharmaceutical compositions provided and described in more detail herein. Such pharmaceutical preparations may be manufactured in a known manner, for example, by means of mixing, granulating, tableting, sugar coating, or film coating processes.

Typically, a pharmaceutical composition as described herein is formulated as a tablet, a troche, a lozenge, an aqueous or oily suspension, a dispersible powder or as granules. A powder may be obtained by e.g. lyophilisation.

Liquid dispersions for oral administration may be syrups, emulsions and suspensions. The syrups may contain as carriers, for example, saccharose or saccharose with glycerine and/or mannitol and/or sorbitol. Suspensions and emulsions may contain a carrier, for example a natural gum, agar, sodium alginate, pectin, methylcellulose, carboxymethylcellulose, or polyvinyl alcohol. Syrups may be formulated to avoid the use of sucrose.

Typically, a pharmaceutical composition as described herein is suitable for oral administration to the subject. Thus, the methods disclosed herein which comprise the use of a pharmaceutical composition as described herein typically comprise orally administering the pharmaceutical composition to the subject.

Typically, the pharmaceutical composition is intended to release the active agent (i.e. the fructosyltransferase) in an appropriate part of the body, where it can be active in converting sucrose. Accordingly, a pharmaceutical composition as described herein may comprise an enteric coating. Any suitable enteric coating material known in the art can be used. Suitable materials include but are not limited to methyl acrylate-methacrylic acid copolymers; cellulose acetate phthalate (CAP); cellulose acetate succinate; hydroxypropyl methyl cellulose (HMPC) and hydroxypropyl methyl cellulose phthalate; hydroxypropyl methyl cellulose acetate succinate (hypromellose acetate succinate; HMPAS); polyvinyl acetate phthalate (PVAP); methyl methacrylate-methacrylic acid copolymers; shellac; cellulose acetate trimellitate; sodium alginate; zein and the like.

The fructosyltransferase is typically stable in the pharmaceutical composition under appropriate storage conditions for extended periods of time. For example, the fructosyltransferase may be stable for in excess of 1 day, 1 month, 1 year, etc, when stored under appropriate conditions. The necessary stability of the fructosyltransferase can be determined based on its application and the form of the composition in which it is provided and can be controlled using methods known in the art, including the use of high purity reagents and storage under appropriate conditions.

Preferred pharmaceutical compositions are sterile and pyrogen free.

Typically, a pharmaceutical composition will contain up to 85 wt % of the fructosyltransferase described herein. It may contain up to 50 wt %, up to 40 wt %, up to up to 20 wt % or up to 10 wt % of the fructosyltransferase.

Typically, a pharmaceutical composition may contain sufficient fructosyltransferase to produce from about 1 to about 100 g, such as from about 2 g to about 50 g, e.g. from about 5 g to about 20 g such as about 10 g of fructooligosaccharides within about 0.5 to 5 hours, such as within about 1 to about 3 hours, e.g. within about 2 hours under physiological conditions. A pharmaceutical composition thus may comprise from about 1 to about 10,000 mg of fructosyltransferase, such as from about 10 to about 1000 mg e.g. about 50 to 500 mg of fructosyltransferase per unit dose.

A pharmaceutical composition may comprise from about 1 mg to about 100 mg such as from about 2 mg to about 50 mg e.g. from about 5 mg to about 20 mg such as from about 7 mg to about 15 mg, e.g. about 10 mg of fructosyltransferase per unit dose.

A pharmaceutical composition may be capable of acting on from about 1% to about 100% (e.g. % w/w or % w/v) e.g. from about 1% to about 80%, such as from about 5% to about 50%, e.g. from about 10% to about 40%, e.g. from about 20 to about 30% of available (e.g. excess) sucrose within about 1 minute to about 1 hour, e.g. within about 10 minutes to about 45 minutes, such as within about 15 minutes to about 30 minutes under physiological conditions. A pharmaceutical composition may thus be capable of converting/incorporating from about 1% to about 100% e.g. from about 1% to about 80%, such as from about 5% to about 50%, e.g. from about 10% to about 40%, e.g. from about to about 30% of available (e.g. excess) fructose into fructooligosaccharides within about 1 minute to about 1 hour, e.g. within about 10 minutes to about 45 minutes, such as within about 15 minutes to about 30 minutes under physiological conditions. In other words a pharmaceutical composition as provided herein may be capable of converting/incorporating from about 1% to about 50% e.g. from about 1% to about 40%, such as from about 5% to about 30%, e.g. from about 10% to about 20%, of the saccharide units present in the available (e.g. excess) sucrose into fructooligosaccharides within about 1 minute to about 1 hour, e.g. within about 10 minutes to about 45 minutes, such as within about 15 minutes to about 30 minutes under physiological conditions.

A pharmaceutical composition may be administered to a subject at any suitable administration frequency. For example, a pharmaceutical composition may be administered at least once per day, such as between about 1 and about 20 times a day, e.g. between about 1 and about 10 times a day, such as between 2 and 5 times a day, e.g. about 3 or 4 times a day.

Also provided herein is a composition described herein, e.g. a pharmaceutical composition described herein, for use in medicine.

Typically, a pharmaceutical composition is used in therapeutic methods. Accordingly, provided herein is a pharmaceutical composition as described herein for use in a method (e.g. a therapeutic method) of reducing fructose uptake; reducing formation of fructose via metabolism of sucrose; reducing glucose uptake and/or reducing formation of glucose via metabolism of sucrose; producing a fructooligosaccharide; suppressing appetite; and/or increasing satiety in a subject. Also provided is a method (e.g. a therapeutic method) of reducing fructose uptake; reducing formation of fructose via metabolism of sucrose; reducing glucose uptake and/or reducing formation of glucose via metabolism of sucrose; producing a fructooligosaccharide; suppressing appetite; and/or increasing satiety in a subject, comprising administering a pharmaceutical composition as described herein to the subject. Still further provided is use of an isolated fructosyltransferase as described herein in the manufacture of a pharmaceutical composition as described herein for the (typically therapeutic) reduction of fructose uptake; reduction of formation of fructose via metabolism of sucrose; reduction of glucose uptake and/or reduction of formation of glucose via metabolism of sucrose; production of a fructooligosaccharide; suppression of appetite; and/or increase in satiety in a subject. Such methods and uses are described in more detail herein.

### Food Compositions and Foodstuffs

Also provided herein are food compositions comprising a fructosyltransferase as described herein. Such food compositions are also referred to herein as foodstuffs. Such compositions may be administered to a subject in accordance with the methods and uses provided herein.

A food composition or foodstuff as described herein typically comprises a fructosyltransferase, e.g. a fructosyltransferase as described herein, and one or more carbohydrates, fats, lipids, flavouring agents, colouring agent, etc.

Food compositions and foodstuffs described herein may comprise:
sugar sources such as corn sugar, dextrose, fructose, glucose, high-fructose glucose syrup, honey, maple syrup, agave syrup, invert sugar, isoglucose, levulose, maltose, molasses, and sucrose;
starch sources such as corn, cassava, sweet potato, wheat (e.g. as flour, e.g. in the form of bread or pasta), potato, sorghum, barley, rice, etc;
fruits such as acal, apple, apricot, avocado, banana, bilberry, blackberry, blackcurrant, blueberry, boysenberry, cherry, cloudberry, crab apple, cranberry, damson, date, dragonfruit, durian, elderberry, fig, goji berry, gooseberry, grape, grapefruit, guava, jackfruit, jujube, kiwifruit, kumquat, lemon, lime, loganberry, lychee, mango, melon, mulberry, nectarine, orange, clementine, mandarine, tangerine, papaya, passionfruit, pawpaw, peach, pear, persimmon, plantain, plum, pineapple, pomegranate, pomelo, quince, raspberry, redcurrant, satsuma, tamarind, yuzu etc;
vegetables such as artichoke, aubergine, asparagus, bean sprouts, beans, chickpeas, lentils, peas, broccoli (calabrese), brussels sprouts, cabbage, cauliflower, celery, endive, fennel, greens such as bok choy, chard (beet greens), collard greens, kale, mustard greens, lettuce, mushrooms, okra, onions, chives, garlic, leek, shallot, scallion, peppers, rhubarb, beetroot, carrot, celeriac, taro, ginger, parsnip, rutabaga, radish, potato, sweet potato, yam, turnip, sweetcorn, squash, courgette, cucumber, tomato, watercress etc.
nuts and seeds such as almonds, Brazil nuts, cashew nuts, hazelnuts, macadamias, pecans, pine nuts, pistachios, walnuts, peanuts, pumpkin seeds, flax seeds, sesame seeds, poppy seeds, sunflower seeds, psyllium seeds and chia seeds.
fats and lipids such as vegetable fats (e.g. cocoa butter, corn oil, sunflower oil, soybean oil, cotton soil, peanut oil, olive oil, canola oil, pumpkin seed oil, safflower oil, grape seed oil, sesame oil bran oil, argan oil, palm oil, linseed oil, coconut oil) and animal fats (e.g. lard, tallow and butterfat, and fish oils such as cod liver oil and salmon oil);
animal products such as meat, fish and eggs.
dyestuffs and other colouring agents such as FD&C Blue No. 1 (brilliant blue FCF), FD&C Blue No. 2 (indigotine), FD&C Green No. 3 (fast green FCF), FD&C Red No. 40 (allura red AC), FD&C Red No. 3 (erythrosine), FD&C Yellow No. 5 (tartrazine), and FD&C Yellow No. 6 (sunset yellow);
flavouring agents such as sweet almond oil, benzaldehyde, DL-menthol, ethyl acetate, ethyl vanillin, L-menthol, methyl salicylate, peppermint oil, peppermint spirit, and vanillin; and
sweeteners such as allulose, acesulfame potassium, aspartame, cyclamate, mogrosides, saccharin, steviol glycosides (stevia), sucralose, and sugar alcohols.

Exemplary foodstuffs include confectionary such as chocolate, desserts such as ice cream, gelato, sorbet, yoghurt, cheesecake, flan, tarts etc; baked goods such as cakes, pastries and pies (both sweet and savory), bread products, etc.

Preferably the foodstuff comprises sucrose.

Typically, a foodstuff as described herein is administered to a subject orally. Thus, the methods disclosed herein which comprise the use of a foodstuff as described herein typically comprise orally administering the foodstuff to the subject.

Typically, the foodstuff is formulated intended to release the active agent (i.e. the fructosyltransferase) in an appropriate part of the body, where it can be active in converting sucrose. For example, the foodstuff may be formulated to release the active fructosyltransferase in the small gastrointestinal tract, e.g. in the small intestine, and/or in the stomach.

The foodstuff may be formulated such that the fructosyltransferase comprised therein is prevented from acting on any sucrose in the foodstuff prior to the foodstuff being consumed. This may be achieved e.g. by encapsulating the fructosyltransferase such that it cannot contact the sucrose prior to the foodstuff being consumed; by physically separating the part of the foodstuff comprising the fructosyltransferase from the part of the foodstuff comprising sucrose, or by formulating the foodstuff to have a condition that is incompatible with significant sucrose conversion prior to the foodstuff being consumed. Alternatively, the fructosyltransferase may be formulated or chosen such that it has low activity outside the body but high activity inside the body, e.g. by selecting or modifying the fructosyltransferase to have a pH- or temperature-dependent activity wherein the active pH or temperature is provided in the body, e.g. in the small intestine, but is not provided by the foodstuff prior to its consumption.

Typically, the food composition or foodstuff comprises only ingredients which are generally recognised as safe (GRAS).

The fructosyltransferase is typically stable in the foodstuff under appropriate storage conditions for extended periods of time. For example, the fructosyltransferase may be stable for in excess of 1 day, 1 month, 1 year, etc, when stored under appropriate conditions. Suitable conditions for the storage of the foodstuff may comprise temperatures such as −25 to −15° C., such as −20 to −18° C. (e.g. for foodstuffs such as ice cream, gelato, sorbet, and other foodstuffs that are sold in frozen form); temperatures such as from about to about 10° C., such as from about 4 to about 7° C. (e.g. for foodstuffs such as yoghurt and chilled desserts that are sold in chilled form); or temperatures such as from about 15 to about 25° C. such as from about 18 to about 20° C. (e.g. for foodstuffs such as chocolate and baked goods e.g. cakes and confectionary that are typically sold at ambient temperature). Suitable conditions for the storage of the foodstuff include under aerobic conditions (e.g. in the presence of air) or anaerobic conditions (e.g. under an inert, e.g. nitrogen environment). Foodstuffs may be provided in the form of a tin, packet, box, pouch or any other suitable container.

The necessary stability of the fructosyltransferase can be determined based on its application and the form of the composition in which it is provided and can be controlled using methods known in the art, including the use of high purity reagents and storage under appropriate conditions.

Typically, a foodstuff will contain up to 10 wt % of the fructosyltransferase described herein. It may contain up to 5 wt %, up to 4 wt %, up to 3 wt %, up to 2 wt % or up to 1 wt % of the fructosyltransferase.

Typically, a foodstuff may contain sufficient fructosyltransferase to produce from about 1 to about 100 g, such as from about 2 g to about 50 g, e.g. from about 5 g to about g such as about 10 g of fructooligosaccharides within about 0.5 to 5 hours, such as within about 1 to about 3 hours, e.g. within about 2 hours under physiological conditions.

A foodstuff thus may comprise from about 0.1 to about 1000 mg of fructosyltransferase, such as from about 1 to about 100 mg e.g. about 10 to about 50 mg of fructosyltransferase per serving. A foodstuff may comprise from about 1 mg to about 100 mg such as from about 2 mg to about 50 mg e.g. from about 5 mg to about 20 mg such as from about 7 mg to about 15 mg, e.g. about 10 mg of fructosyltransferase per serving.

A foodstuff may contain sufficient fructosyltransferase to act on from about 1% to about 100% (e.g. % w/w or % w/v) e.g. from about 1% to about 80%, such as from about 5% to about 50%, e.g. from about 10% to about 40%, e.g. from about 20 to about 30% of available sucrose (e.g. of the sucrose molecules in the foodstuff) within about 1 minute to about 1 hour, e.g. within about 10 minutes to about 45 minutes, such as within about 15 minutes to about 30 minutes under physiological conditions. A foodstuff may thus contain sufficient fructosyltransferase be capable of converting/incorporating from about 1% to about 100% e.g. from about 1% to about 80%, such as from about 5% to about 50%, e.g. from about 10% to about 40%, e.g. from about 20 to about 30% of available fructose (e.g. of available fructose in the foodstuff) into fructooligosaccharides within about 1 minute to about 1 hour, e.g. within about 10 minutes to about 45 minutes, such as within about 15 minutes to about 30 minutes under physiological conditions. In other words a foodstuff as provided herein may contain sufficient fructosyltransferase to be capable of converting/incorporating from about 1% to about 50% e.g. from about 1% to about 40%, such as from about 5% to about 30%, e.g. from about 10% to about 20%, of the saccharide units present in the available sucrose (e.g. in the sucrose in the foodstuff) into fructooligosaccharides within about 1 minute to about 1 hour, e.g. within about 10 minutes to about 45 minutes, such as within about 15 minutes to about 30 minutes under physiological conditions.

A subject may consume a foodstuff as described herein between about 1 and about times a day, such as between 2 and 5 times a day, e.g. about 3 or 4 times a day.

Typically, a foodstuff is consumed in a non-therapeutic context. Accordingly, provided herein is use of a foodstuff as described herein in a method (e.g. a non-therapeutic method) of reducing fructose uptake; reducing formation of fructose via metabolism of sucrose; reducing glucose uptake and/or reducing formation of glucose via metabolism of sucrose; producing a fructooligosaccharide; suppressing appetite; and/or increasing satiety in a subject. Also provided is a method (e.g. a non-therapeutic method) of reducing fructose uptake; reducing formation of fructose via metabolism of sucrose; reducing glucose uptake and/or reducing formation of glucose via metabolism of sucrose; producing a fructooligosaccharide; suppressing appetite; and/or increasing satiety in a subject, comprising administering a foodstuff as described herein to the subject. Further provided is a foodstuff as described herein for use in a method (e.g. a non-therapeutic method) of reducing fructose uptake; reducing formation of fructose via metabolism of sucrose; reducing glucose uptake and/or reducing formation of glucose via metabolism of sucrose; producing a fructooligosaccharide; suppressing appetite; and/or increasing satiety in a subject. Still further provided is use of an isolated fructosyltransferase as described herein for use in the manufacture of a foodstuff as described herein for the (typically non-therapeutic) reduction of fructose uptake; reduction of formation of fructose via metabolism of sucrose; reduction of glucose uptake and/or reduction of formation of glucose via metabolism of sucrose; production of a fructooligosaccharide; suppression of appetite; and/or increase in satiety in a subject. Such methods and uses are described in more detail herein.

### Therapeutic and Non-Therapeutic Efficacy

The fructosyltransferases described herein are useful in reducing fructose uptake and metabolism of sucrose to form glucose and fructose. As such, they can be used in controlling the energy taken up by a subject following consumption of food such as sugar.

Accordingly, as described in more detail herein, provided is an in vivo method of reducing fructose uptake in a subject, the method comprising administering to the subject an isolated fructosyltransferase. Typically the method is a non-therapeutic method. Typically the non-therapeutic use of an isolated fructosyltransferase in accordance with the methods provided herein does not comprise treatment of the human or animal body by surgery or therapy. Also provided is an isolated fructosyltransferase for use in reducing fructose uptake in vivo in a subject. Further provided is the use of an isolated fructosyltransferase for the manufacture of an agent for reducing fructose uptake in vivo in a subject.

As also described in more detail herein, provided is an in vivo method of reducing the formation of fructose via metabolism of sucrose in a subject, the method comprising administering to the subject an isolated fructosyltransferase. Typically the method is a non-therapeutic method. Typically the non-therapeutic use of an isolated fructosyltransferase in accordance with the methods provided herein does not comprise treatment of the human or animal body by surgery or therapy. Also provided is an isolated fructosyltransferase for use in reducing the formation of fructose via metabolism of sucrose in vivo in a subject. Further provided is the use of an isolated fructosyltransferase for the manufacture of an agent for reducing the formation of fructose via metabolism of sucrose in vivo in a subject.

However, the fructosyltransferases described herein also have other therapeutic and non-therapeutic uses.

In one aspect, administration of an isolated fructosyltransferase can be used to suppress a subject's appetite and/or increase satiety. Exogenous inulin has previously been shown to have beneficial effects on weight management through appetite control (e.g. see Guess et al, Nutrition & Metabolism 12 36 (2015) accessible at https://doi.org/10.1186/s12986-015-0033-2). The inventors have recognised that similar beneficial effects will arise from the production of inulin and related fructooligosaccharides in vivo in accordance with the methods provided herein. Without being bound by theory, one mechanism proposed for the suppression of appetite is the fructooligosaccharides-stimulated production of peptide YY. Peptide YY is also known as peptide tyrosine tyrosine, and is a short (36-amino acid) peptide released from cells in the ileum and colon in response to feeding. In the blood, gut, and other elements of periphery, PYY acts to reduce appetite; similarly, when injected directly into the central nervous system, PYY is also anorexigenic. (Woods S. C.; D'Alessio D. A. (2008). “Central control of body weight and appetite”. J Clin Endocrinol Metab. 93 (11 Suppl 1): S37-50.)

Accordingly, provided herein is a method of suppressing a subject's appetite, comprising administering to the subject an isolated fructosyltransferase or a composition comprising an isolated fructosyltransferase as described herein. Also provided is a method of increasing a subject's satiety, comprising administering to the subject an isolated fructosyltransferase or a composition comprising an isolated fructosyltransferase as described herein. Typically such methods are non-therapeutic methods. Also provided is an isolated fructosyltransferase or a composition comprising an isolated fructosyltransferase as described herein for use in suppressing a subject's appetite. An isolated fructosyltransferase or a composition comprising an isolated fructosyltransferase as described herein for use in increasing a subject's satiety is also provided. Further provided is the use of an isolated fructosyltransferase or a composition comprising an isolated fructosyltransferase as described herein in the manufacture of an agent for suppressing a subject's appetite. The use of an isolated fructosyltransferase or a composition comprising an isolated fructosyltransferase as described herein in the manufacture of an agent for increasing a subject's satiety is also provided.

The fructosyltransferase may be administered to a subject for cosmetic purposes. Such purposes may comprise the non-therapeutic administration of the fructosyltransferase to a subject desiring the improvement of their body appearance. For example, in one embodiment provided herein is a method (e.g. a non-therapeutic and/or cosmetic method) of improving the bodily appearance of a subject comprising orally administering to the subject an isolated fructosyltransferase or a composition comprising an isolated fructosyltransferase as described herein in such an amount to decrease the appetite and/or increase the satiety of the subject, and repeating said administration until a cosmetically-desirable loss of body weight has occurred.

The composition used in such methods and uses may be a nutraceutical or pharmaceutical composition or a foodstuff as described herein. Typically, the isolated fructosyltransferase or composition is administered to the subject orally.

In another aspect, administration of an isolated fructosyltransferase can be used to treat or prevent metabolic syndrome. Metabolic syndrome is a clustering of at least three of the following five medical conditions: abdominal obesity, high blood pressure, high blood sugar, high serum triglycerides, and low serum high-density lipoprotein (HDL). Metabolic syndrome is associated with the risk of developing cardiovascular disease and type 2 diabetes. Metabolic syndrome can be diagnosed by the presence of any one of diabetes mellitus, impaired glucose tolerance, impaired fasting glucose or insulin resistance, AND two of the following:
Blood pressure≥140/90 mmHg
Dyslipidemia: triglycerides (TG)≥1.695 mmol/L and HDL cholesterol≤0.9 mmol/L (male),≤1.0 mmol/L (female)
Central obesity: waist:hip ratio>0.90 (male);>0.85 (female), or BMI>30 kg/m²
Microalbuminuria: urinary albumin excretion ratio≥20 μg/min or albumin:creatinine ratio≥30 mg/g.

Excess sucrose consumption and metabolism has been associated with metabolic syndrome (e.g. see Malik et al, Diabetes Care 2010 33(11) 2477-2483). Without being bound by theory, it is believed that by reducing the concentration of sucrose available for metabolism, metabolic syndrome can be addressed by administration of isolated fructosyltransferase in accordance with the methods provided herein.

Accordingly, provided herein is an isolated fructosyltransferase, or a pharmaceutically acceptable composition comprising an isolated fructosyltransferase, for use in treating or preventing metabolic syndrome in a subject in need thereof. Also provided is a method of treating or preventing metabolic syndrome in a subject in need thereof, the method comprising administering an isolated fructosyltransferase, or a pharmaceutically acceptable composition comprising an isolated fructosyltransferase to the subject. Further provided is the use of an isolated fructosyltransferase, or a pharmaceutically acceptable composition comprising an isolated fructosyltransferase, in the manufacture of a medicament for treating metabolic syndrome in a subject. The composition used in such methods and uses may be a nutraceutical or pharmaceutical composition described herein. Typically, the isolated fructosyltransferase or composition is administered to the subject orally.

When applied in non-therapeutic methods and uses, the fructosyltransferase may be administered to a subject who is not suffering from and/or is not at risk of suffering from metabolic syndrome (e.g. is not suffering from and/or is not at risk of suffering from abdominal obesity, high blood pressure (e.g. ≥140/90 mmHg), high blood sugar, high serum triglycerides (e.g. ≥1.695 mmol/L), low serum high-density lipoprotein (HDL) (e.g. ≤0.9 mmol/L (male), ≤1.0 mmol/L (female)), cardiovascular disease, type 2 diabetes, diabetes mellitus, impaired glucose tolerance, impaired fasting glucose or insulin resistance, elevated blood pressure, dyslipidemia; central obesity (e.g. waist:hip ratio>(male); >0.85 (female), or BMI>30 kg/m²) and/or microalbuminuria (e.g. urinary albumin excretion ratio≥20 μg/min or albumin:creatinine ratio≥30 mg/g)).

Provided herein is a method of maintaining the health of a healthy subject, comprising administering to the subject an isolated fructosyltransferase, optionally in the form of a composition as described herein. Also provided is the use of an isolated fructosyltransferase, optionally in the form of a composition described herein, for maintaining the health of a healthy individual.

Excess sucrose consumption and metabolism has also been associated directly with obesity. A subject may be considered obese if they have a body mass index (BMI) (defined by dividing the subject's weight by the square of their height) in excess of 30 kg/m². A subject may be considered overweight if they have a BMI of between about 25 and 30 kg/m². Without being bound by theory, it is believed that by reducing the concentration of sucrose available for metabolism, obesity can be addressed by administration of isolated fructosyltransferase in accordance with the methods provided herein. As used herein, addressing or treating obesity may include addressing or treating a subject who has a BMI of in excess of 30 kg·m² or who has a BMI of between 25 and 30 kg/m².

Accordingly, provided herein is an isolated fructosyltransferase, or a pharmaceutically acceptable composition comprising an isolated fructosyltransferase, for use in treating or preventing obesity in a subject in need thereof. Also provided is a method of treating or preventing obesity in a subject in need thereof, the method comprising administering an isolated fructosyltransferase, or a pharmaceutically acceptable composition comprising an isolated fructosyltransferase to the subject. Further provided is the use of an isolated fructosyltransferase, or a pharmaceutically acceptable composition comprising an isolated fructosyltransferase, in the manufacture of a medicament for treating obesity in a subject. The composition used in such methods and uses may be a nutraceutical or pharmaceutical composition described herein. Typically, the isolated fructosyltransferase or composition is administered to the subject orally.

When applied in non-therapeutic methods and uses, the fructosyltransferase may be administered to a subject who is not overweight and/or is not obese. For example, the fructosyltransferase may be administered in the non-therapeutic methods and uses provided herein to a subject with a BMI of less than about 30 kg/m², e.g. less than about 25 kg/m².

Diabetes is a further disorder associated with excess sucrose levels in vivo. Diabetes is commonly linked with insulin deficiency. Type 1 diabetes results from reduced insulin production by the pancreas due to loss of beta cells caused by autoimmune responses. Type 2 diabetes arises from insulin resistance. Gestational diabetes is a further form of diabetes. Without being bound by theory, it is believed that administering an isolated fructosyltransferase in accordance with the methods provided herein can reduce sucrose levels in vivo and thus have beneficial effects in treating or preventing diabetes. Administering an isolated fructosyltransferase in accordance with the methods provided herein can also beneficially reduce glucose levels in vivo as described herein.

Accordingly, provided herein is an isolated fructosyltransferase, or a pharmaceutically acceptable composition comprising an isolated fructosyltransferase, for use in treating or preventing diabetes in a subject in need thereof. Also provided is a method of treating or preventing diabetes in a subject in need thereof, the method comprising administering is an isolated fructosyltransferase, or a pharmaceutically acceptable composition comprising an isolated fructosyltransferase to the subject. Further provided is the use of an isolated fructosyltransferase, or a pharmaceutically acceptable composition comprising an isolated fructosyltransferase, in the manufacture of a medicament for treating diabetes in a subject. Often, the diabetes is type 2 diabetes. The composition used in such methods and uses may be a nutraceutical or pharmaceutical composition described herein. Typically, the isolated fructosyltransferase or composition is administered to the subject orally.

When applied in non-therapeutic methods and uses, the fructosyltransferase may be administered to a subject who is not suffering from and/or is not at risk of suffering from diabetes. The fructosyltransferase may be administered to a subject having a fasting blood glucose level of from about 4 mM to about 5.5 mM or about 6 mM and/or a post-prandial (e.g. 90 minutes post-prandial) blood glucose level of under about 7.8 mM. The fructosyltransferase may not, in some embodiments, be administered to a subject with a fasting blood glucose level of 4-7 mM, e.g. more than about 6 mM, and/or a post-prandial (e.g. 90 minutes post-prandial) blood glucose level of more than 7.8 mM.

Still a further condition associated with excess sucrose levels in vivo is non-alcoholic fatty liver disease. High fructose levels from sucrose consumption promotes fat accumulation in the liver by stimulating de novo lipogenesis in the liver and reducing the beta-oxidation of fat. In addition, fructokinases rapidly metabolize fructose leading to decreased intracellular ATP levels in the liver, which may increase oxidative stress impairing protein synthesis and mitochondrial liver function. Administering an isolated fructosyltransferase in accordance with the disclosed methods reduces fructose levels taken up by the body and thus can have beneficial effects in treating or preventing non-alcoholic fatty liver disease.

Accordingly, provided herein is an isolated fructosyltransferase, or a pharmaceutically acceptable composition comprising an isolated fructosyltransferase, for use in treating or preventing non-alcoholic fatty liver disease in a subject in need thereof. Also provided is a method of treating or preventing non-alcoholic fatty liver disease in a subject in need thereof, the method comprising administering is an isolated fructosyltransferase, or a pharmaceutically acceptable composition comprising an isolated fructosyltransferase to the subject. Further provided is the use of an isolated fructosyltransferase, or a pharmaceutically acceptable composition comprising an isolated fructosyltransferase, in the manufacture of a medicament for treating non-alcoholic fatty liver disease in a subject. The composition used in such methods and uses may be a nutraceutical or pharmaceutical composition described herein. Typically, the isolated fructosyltransferase or composition is administered to the subject orally.

When applied in non-therapeutic methods and uses, the fructosyltransferase may be administered to a subject who is not suffering from and/or is not at risk of suffering from non-alcoholic fatty liver disease.

Yet another condition amenable to treatment using an isolated fructosyltransferase is constipation. Constipation is among the most common health impediments especially in elderly populations. Inulin is non-digestible by humans and its fermentation in the colon can lead to increased bacterial cell mass and a higher water content of digesta, which aids bowel function. Accordingly, administering an isolated fructosyltransferase in accordance with the disclosed methods promotes inulin production and can thus have beneficial effects in treating or preventing constipation.

Accordingly, provided herein is an isolated fructosyltransferase, or a pharmaceutically acceptable composition comprising an isolated fructosyltransferase, for use in treating or preventing constipation in a subject in need thereof. Also provided is a method of treating or preventing constipation in a subject in need thereof, the method comprising administering is an isolated fructosyltransferase, or a pharmaceutically acceptable composition comprising an isolated fructosyltransferase to the subject. Further provided is the use of an isolated fructosyltransferase, or a pharmaceutically acceptable composition comprising an isolated fructosyltransferase, in the manufacture of a medicament for treating constipation in a subject. The composition used in such methods and uses may be a nutraceutical or pharmaceutical composition described herein. Typically, the isolated fructosyltransferase or composition is administered to the subject orally.

When applied in non-therapeutic methods and uses, the fructosyltransferase may be administered to a subject who is not suffering from and/or is not at risk of suffering from constipation.

The methods and uses provided herein (particularly the therapeutic methods and uses described herein) may comprise administering the isolated fructosyltransferase or composition comprising an isolated fructosyltransferase together with one or more additional therapies or compositions. For example, the fructosyltransferase or composition may be administered together with conventional therapies for treating obesity. Such agents include orlistat, lorcaserin, liraglutide, phentermine—topiramate, metformin and naltrexone—bupropion. Where separately formulated, the two agents may be administered simultaneously or separately. They may be provided in the form of a kit, optionally together with instructions for their administration.

Alternatively or additionally, the fructosyltransferase or compositions provided herein may be administered to a subject who is or has been also treated surgically, e.g. via gastric banding. For example, the subject may have received laparoscopic adjustable gastric banding, Roux-en-Y gastric bypass, vertical-sleeve gastrectomy, or biliopancreatic diversion.

As described herein, an isolated fructosyltransferase as provided herein, or a composition comprising an isolated fructosyltransferase, can be administered to any suitable subject.

In one aspect, the subject is a mammal, in particular a human. However, it may be non-human. Preferred non-human animals include, but are not limited to, primates, such as marmosets or monkeys, commercially farmed animals, such as horses, cows, sheep or pigs, and pets, such as dogs, cats, mice, rats, guinea pigs, ferrets, gerbils or hamsters.

A subject may be overweight or obese. For example, a human subject may have a BMI of in excess of 25 kg/m²; in excess of 30 kg/m²; or in excess of 35 kg/m′. A subject may be male or female. A subject may be aged from about 10 to about 80, such as from about 16 or about 18 to about 65; such as from about 20 to about 60, e.g. from about 25 to about 55, such as from about 30 to about 50. A subject may be of any racial or genetic background.

An agent described herein can be administered to the subject in order to prevent the onset or reoccurrence of one or more pathological symptoms, e.g. symptoms of obesity or metabolic syndrome. This is prophylaxis. In this embodiment, the subject can be asymptomatic. The subject is typically one that is at risk of obesity or metabolic syndrome. A prophylactically effective amount of the agent or formulation is administered to such a subject. A prophylactically effective amount is an amount which prevents the onset of one or more symptoms of obesity or metabolic syndrome.

An agent described herein can be administered to the subject in order to treat one or more pathological symptoms, e.g. symptoms or obesity or metabolic syndrome. In this embodiment, the subject is typically symptomatic. A therapeutically effective amount of the agent or formulation is administered to such a subject. A therapeutically effective amount is an amount effective to ameliorate one or more symptoms of the disorder.

The agent (i.e. the isolated fructosyltransferase or composition comprising the isolated fructosyltransferase) may be administered in a variety of dosage forms. Usually, it is administered orally, for example as tablets, troches, lozenges, aqueous or oily suspensions, dispersible powders or granules. Such formulations are described in more detail herein.

However, for some applications the agent may also be administered parenterally, whether subcutaneously, intravenously, intramuscularly, intrasternally, transdermally or by infusion techniques. Solutions for inhalation, injection or infusion may contain as carrier, for example, sterile water or preferably they may be in the form of sterile, aqueous, isotonic saline solutions. Pharmaceutical compositions suitable for delivery by needleless injection, for example, transdermally, may also be used.

The agent may also be administered as a suppository.

The agent may in some circumstances be administered via inhalation. The agent may be formulated for inhaled (aerosolised) administration as a solution or suspension. The compound, composition or combination of the invention may be administered by a metered dose inhaler (MDI) or a nebulizer such as an electronic or jet nebulizer. Alternatively, the compound, composition or combination of the invention may be formulated for inhaled administration as a powdered drug, such formulations may be administered from a dry powder inhaler (DPI). When formulated for inhaled administration, the compound, composition or combination of the invention may be delivered in the form of particles which have a mass median aerodynamic diameter (MMAD) of from 1 to 100 μm, preferably from 1 to 50 μm, more preferably from 1 to 20 μm such as from 3 to 10 μm, e.g. from 4 to 6 μm. When the compound, composition or combination of the invention is delivered as a nebulized aerosol, the reference to particle diameters defines the MMAD of the droplets of the aerosol. The MMAD can be measured by any suitable technique such as laser diffraction.

In use, a therapeutically or prophylactically effective amount of the agent is administered to a subject. The dose may be determined according to various parameters, especially according to the agent used; the age, weight and condition of the subject to be treated; the route of administration; and the required regimen. A physician or dietician will be able to determine the required route of administration and dosage for any particular subject. A typical daily dose is from about 0.01 to 100 mg per kg, preferably from about mg/kg to 50 mg/kg, e.g. from about 1 to 10 mg/kg of body weight, according to the activity of the specific agent or inhibitor, the age, weight and conditions of the subject to be treated, the type and severity of the disease and the frequency and route of administration. Preferably, daily dosage levels are from 5 mg to 2 g.

In both therapeutic and non-therapeutic methods and uses, the amount of the agent to be administered is sufficient to convert a physiologically useful amount of sucrose to fructooligosaccharides. For example, although the volume of the small intestine varies considerably between subjects, a typical volume is in the region of 150 to 250 mL, such as around 180 mL. Sufficient agent may be administered to result in a small intestinal concentration of around 10-100 μg/mL such as from about 20 to about 70 μg/mL e.g. about 50 μg/mL. For example, a dose of from about 1 mg to about 100 mg such as from about 2 mg to about 50 mg e.g. from about 5 mg to about 20 mg such as about 10 mg may be administered.

It is to be understood that although particular embodiments, specific configurations as well as materials and/or molecules, have been discussed herein for methods according to the present invention, various changes or modifications in form and detail may be made without departing from the scope and spirit of this invention. The following examples are provided to better illustrate particular embodiments, and they should not be considered limiting the application. In particular, there are many assays for assessing formation of fructooligosacharides, activity of enzymes, etc, and so a negative result in any specific assay is not determinative.

### EXAMPLES

In the examples, references to SEQ ID NOs: 4, 5, 6, 7, 9 and 10 refer to the polypeptides of 4a, 5a, 6a, 7a, 9a, and 10a. In other words, references to SEQ ID NOs: 4, 5, 6, 7, 9, and 10 refer to the polypeptide sequences minus the signal peptide.

### Example 1: Growth of Cells Expressing Fructosyltransferase Candidates

E. coli BL21(DE3) lac^{IQ} cells transformed with pAVE1 (pET28a(+) derived expression plasmid) producing the proteins of SEQ ID NOs: 4, 5 or 7 were grown in LB media at 37° C. until OD₆₀₀ of 0.6 was reached. The cultures were induced with 0.1 mM IPTG, moved to 28° C., and incubated for another 12 h.
E. coli BL21(DE3) lac^{IQ} cells transformed with pAVE1 producing the proteins of SEQ ID NOs: 1, 2, 3, 6, 8, 9 or 10 were grown in complex auto-induction media at 28° C. for 26 h min.

### Example 2: Purification of Candidate Enzymes

Candidate enzymes were purified using the commercially available Protino Ni-IDA 2000 kit. 200 mL (SEQ ID NOs 1/2/3/5/6/8/9/10) or 400 mL (SEQ ID NOs: 4/7) cultures were grown as described above, harvested by centrifugation at 4,500 RPM for 15 min at 4° C. Cell pellets were thawed and resuspended in 5 mL LEW Buffer (50 mM NaH₂PO₄, 300 mM NaCl, adjusted to pH 8 using NaOH) per gram of cell pellet. Phenylmethylsulfonyl fluoride (PMSF) was added to 0.2 mM. The cell suspensions were sonicated for five cycles of 15 s pulse and 15 s break at 70% amplitude. Lysates were cleared by centrifugation at RPM for 30 min at 4° C. and filtered through a 0.2 μm membrane before adding the supernatant to LEW buffer-equilibrated Protino Ni-IDA 2000 columns. The protein-bound columns were washed with 2×4 mL LEW washing buffer before eluting with 3×3 mL elution buffer (50 mM NaH₂PO₄, 300 mM NaCl, 250 mM imidazole, adjusted pH to 8.0 using NaOH). Elutions were buffer exchanged by five rounds of centrifugation using Amicon centrifugal filters into 50 mM potassium phosphate buffer pH 7.0. Each centrifugation round was performed at 4,500 RPM for 20 min at 4° C. Finally, purified proteins were supplemented to 10% glycerol and stored at −20° C. Purity was assayed by sodium dodecyl sulphate polyacrylamide gel electrophoresis (SDS-PAGE). Concentrations of purified proteins were quantified by gel densitometry using ImageJ with three defined bovine serum albumin (BSA) standards.

### Example 3: Initial Fructosyltransferase Activity Screen

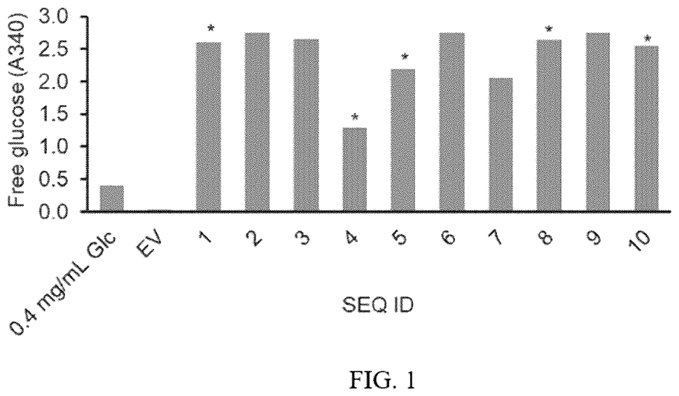

Fructosyltransferases (FTases) (inulosucrases or levansucrases) of SEQ ID NOs 1-10 were selected for analysis. FTases were initially screened for activity in cell lysate. Sucrose digestion was assayed in cell lysates as a fast way to determine enzyme activity. E. coli BL21(DE3) lac^{IQ} transformed with pAVE1 encoding a FTase were grown in 500 μl LB, minimal auto-induction or complex auto-induction media in deep-well plates at 20, 28 or 37° C. for 6, 12 or 24 h (post-induction, auto-induction cultures reached OD₆₀₀=0.6 after approximately 135 min). Cells were harvested by centrifugation at 4,500 RPM for 15 min at 4° C. The cell pellets were resuspended in 160 μl SIF(−/−) buffer before adding lysozyme to a final concentration of 1 mg/mL. Cell suspensions were incubated at 37° C. min before centrifugation at 4,500 RPM for 30 min at 4° C. 75 μl soluble lysate was mixed with 25 μl sucrose and incubated for 2 h at 37° C. Reactions were quenched by incubation at 95° C. for 10 min. Free glucose in 10 μl reaction mixture was determined as described below. Samples of SEQ ID NOs: 1, 4, 5, 8, and 10 were diluted 1:100.
FTases were expressed in E. coli in several combinations of expression conditions. The soluble cell lysate fraction was incubated with sucrose in simulated intestinal conditions without pancreatin or bile salts (SIF −/−). Activity was monitored by release of free glucose (FIG. 1). All of SEQ ID NOs 1-10 showed strong activity releasing more substantially more than 0.4 mg/mL glucose. Results are shown in FIG. 1. SEQ ID NOs 2, 4, 7 and 8 were loaded in duplicate (2a/b, 4a/b, 7a/b, 8a/b).

### Example 4: Refined Expression and Purification of Fructosyltransferases

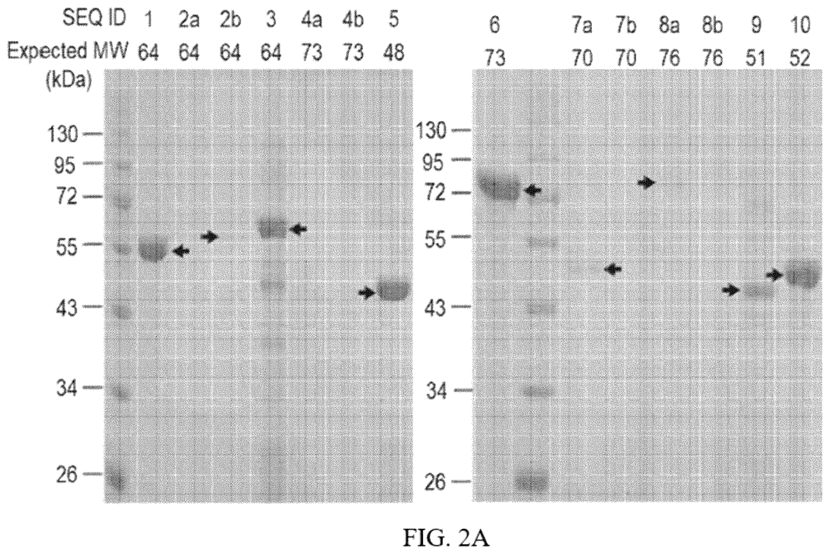

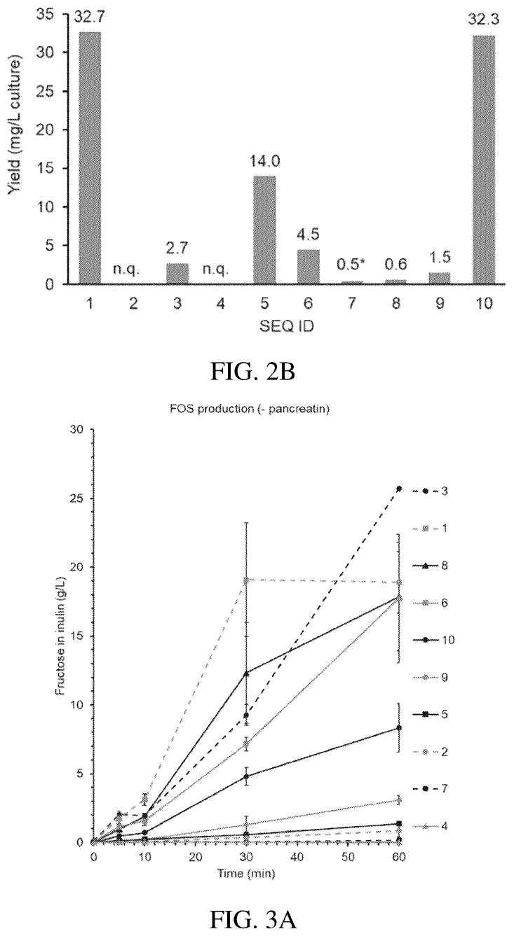

Optimal expression conditions identified in the initial screen were used for each enzyme. FTases were purified using immobilised nickel affinity chromatography (FIG. 2). Quantifiable yields of FTases varied from 0.5 mg/L culture (SEQ ID NO: 7) to 32.7 mg/L culture (SEQ ID NO: 1). The yields of the proteins of SEQ ID NOs: 2 and 4 were not quantifiable by gel densitometry. The purified band for SEQ ID NO: 7 was of lower molecular weight (MW, −50 kDa) than expected (70 kDa). SAS-PAGE gels and quantified expression levels are shown in FIG. 2.

### Example 5: Characterization of Purified Inulosucrase Activity in Simulated Intestinal Fluid

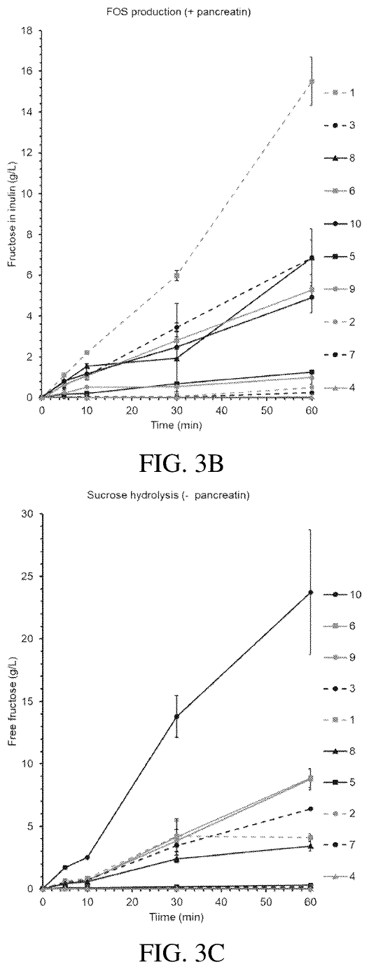

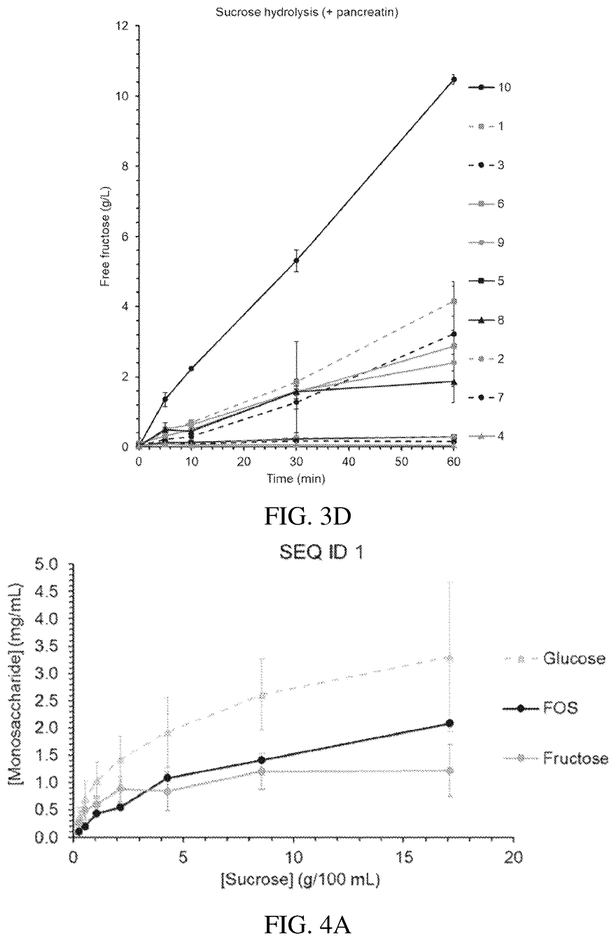

The activity of purified FTases was assessed in simulated intestinal fluid containing bile acids without (SIF+/−) and with (SIF+/+) pancreatin (Brodkorb, A. et al. (2019) Nature Protocols, 14(4), pp. 991-1014) using 500 mM sucrose as substrate. FTase activity was assessed by release of free total monosaccharides (glucose and fructose) and free glucose. The difference between free glucose and free fructose was used to monitor fructooligosaccharide (FOS) production (FIG. 3).
FTases were analysed in simulated intestinal fluid (SIF, 6.8 mM KCl, 0.8 mM, KH₂PO₄, 123.4 mM NaCl, 0.33 mM MgCl₂ (H₂O)₆, 8.4 mM HCl, 0.6 mM CaCl₂ (H₂O)₂), 10 mM bile acids (B8756, Sigma, average molecular weight=422.6 g/mol) with (SIF+/+) and without (SIF+/−) 30 mg/mL pancreatin. 30 mg/mL pancreatin was validated to contain 100 U/mL trypsin activity, where 1 U hydrolyses 1 μmol of p-toluene-sulfonyl-L-arginine methyl ester (TAME) per min at pH 8.1 at 25° C. in 46 mM Tris-HCl 11.5 mM CaCl₂. The composition of the SIF buffers are adapted from the INFOGEST 2.0 protocol (Brodkorb et al., 2019) by accounting for the consecutive dilution of the salivary and gastric phase into the intestinal phase. Amylase, gastric lipase, and pepsin were excluded because the substrate does not include starch or lipids, and the pH is above the complete inhibitory level of pepsin (Johnston, N. et al. (2007) The Laryngoscope, 117(6), pp. 1036-1039; Piper, D. W. and Fenton, B. H. (1965) Gut, 6(5), pp. 506-508.). Each reaction was composed of 60 μl 100 μg/mL inulosucrase and 540 μl of 1.11× SIF buffers. Reaction volumes were incubated at 37° C. and 100 μl samples were collected after 5, 10, 30, and 60 min. Samples were inactivated at 95° C. for 10 min and hydrolysis and transglycosylation rates were determined as described below.
The rate of hydrolysis and transfructosylation of inulosucrases in simulated intestinal fluid was determined essentially as described in Salim, A. S. et al. (2017) ‘Enzymatic synthesis of fructo-oligosaccharides by recombinant levansucrase from Leuconostoc mesenteroides Lm17’, Bulgarian Chemical Communications, Volume 49, Special Issue D (pp. 259-264). Briefly, total free D-glucose and D-fructose (total monosaccharide) was measured in an enzymatic assay using hexokinase, glucose-6P-dehydrogenase and phospho-glucose isomerase (K-FRUGL, Megazyme International Ireland Ltd., Wicklow, Ireland) according to the manufacturer's instructions. Colorimetric measurements were performed using a ClarioStar plus (BMG) spectrophotometer. Free glucose (excluding fructose) was measured by omitting phosphoglucose isomerase. The linearity of the assay was determined to be 0.01-0.8 g/L glucose. Accordingly, samples were diluted to <0.8 g/L glucose. Free fructose concentration was calculated from the difference of combined free D-glucose and D-fructose (total monosaccharide) and free D-glucose alone. Hydrolysis of sucrose (1) yields free fructose and glucose whereas transfructosylation (2) results in fructose incorporated into the inulin fibre and free glucose:

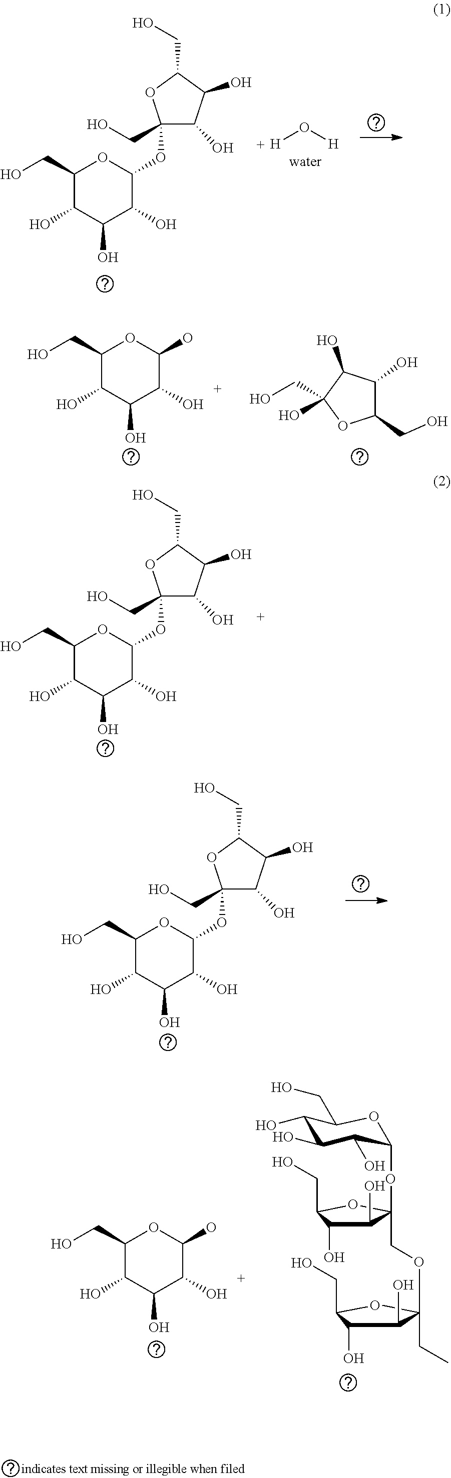

Therefore, the amount of free fructose is a direct measure of the portion of hydrolysed sucrose (non-transfructosylated).

frc_{free}=mon−glc (3)

Every hydrolysis and transfructosylation reaction releases glucose. By subtracting the free fructose from the total amount of glucose, the amount of glucose originating from transfructosylation is obtained. As sucrose is a 1:1 stoichiometry of glucose:fructose, the glucose attributed to transfructosylation is a direct measure of the fructose incorporated into the FOS.

frc_{FOS}=glc−frc_{free} (4)

In order to determine the transfructosylation ratio,

$$Transfructosylation ( \% ) = 100 \times \frac{{frc}_{FOS}}{glc} \qquad ( 5 )$$

$$Hydrolysis ( \% ) = 100 \times \frac{{frc}_{free}}{glc} \qquad ( 6 )$$

In the absence of pancreatin four FTases (SEQ ID NOs: 1, 3, 6 and 8) incorporated more than 19% (17.7 g/L) of total available fructose (500 mM free fructose=90 g/L) into FOS (FIG. 3A). In the presence of pancreatin the activity of all FTases was reduced. SEQ ID NO: 1 showed the smallest reduction in activity; SEQ ID NO: 1 produced 18% less FOS by 60 min in the presence of pancreatin (15.5 g/L) than in the absence of pancreatin (18.9 g/L). The levansucrase of SEQ ID NO: 10 was active in the presence and absence of pancreatin but exhibits a high ratio of hydrolysis to transfructosylation.

### Example 6: GRAVY Scores for Fructosyltransferases of SEQ ID NOs: 1-8

GRAVY scores were determined for each of the proteins of SEQ ID NOs: 1-8 using the tool accessible at https://www.bioinformatics.org/sms2/protein_gravy.html. Results are shown in the following table.

| SEQ ID NO: | GRAVY score |
|---|---|
| 1 | −0.601 |
| 2 | −0.663 |
| 3 | −0.636 |
| 4 | −0.611 |
| 5 | −0.604 |
| 6 | −0.628 |
| 7 | −0.205 |
| 8 | −0.497 |

### Example 7: Activity of FTases at Low Sucrose Concentrations

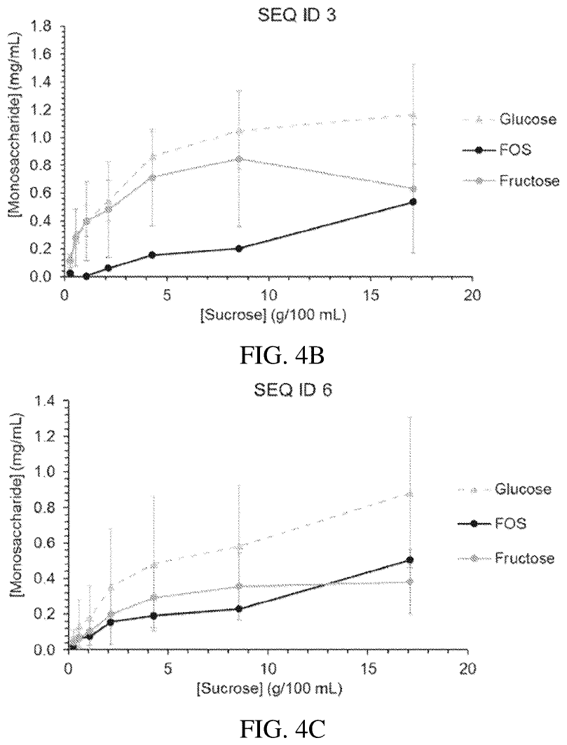

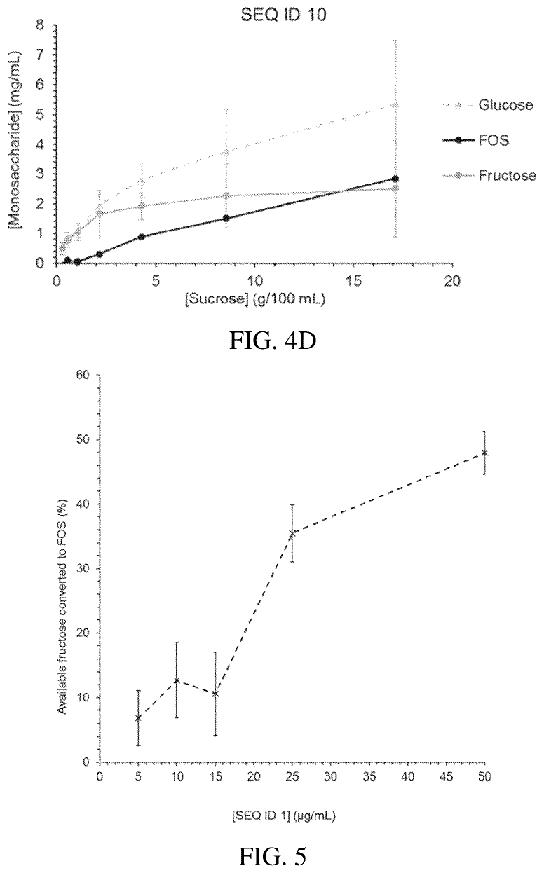

Activity of FTases at low sucrose concentrations is typically beneficial for optimal performance in vivo. Specifically, it can be important to maintain a high ratio of transfructosylation compared to hydrolysis (T/H), especially at lower sucrose concentrations which favours hydrolysis.
The activity of SEQ ID NOs 1, 3, 6 and 8 at a range of physiologically relevant sucrose concentrations was tested. The experiment was performed in simulated duodenal conditions including pancreatin, fresh porcine bile and at approximately pH 5.5 (Houghton et al. Food Chemistry. 2014 15; 151:352-7). 5 μg/mL FTase was incubated in simulated duodenal conditions with sucrose for 30 min at 37° C. Concentrations of free glucose and fructose were determined as described above.
Results are shown in FIG. 4. All FTases tested retained useful transfructosylation activity at low sucrose concentrations. For all FTases tested the T/H decreased as the sucrose concentration decreased. SEQ ID NOs 1 and 6 maintained particularly high T/H at low sucrose concentrations. For example, the T/H of SEQ ID NO 1 was 0.63 at 17.2% sucrose, 0.42 at 1% and 0.29 at 0.5%.
This example confirms that significant sucrose conversion can be achieved even at physiologically-relevant sucrose concentrations using the FTases described herein.

### Example 8: Enzyme Concentration Dependence of Sucrose Conversion

Higher concentrations of FTase were tested in simulated duodenal conditions (Houghton et al. Food Chemistry. 2014 15; 151:352-7) with 125 mM sucrose (4.2% or 4.2 g/100 mL) for 30 min at 37° C. Production of FOS was inferred from release of free glucose and fructose as described above.
Results are shown in FIG. 5. A linear increase in FOS production with increasing concentrations of FTase was observed. 50 μg/mL FTase (SEQ ID NO 1) converted 47.9±3.3% of available fructose into FOS in 30 min.
This example confirms that significant and rapid sucrose conversion can be achieved using practically-accessible amounts of the FTases described herein.

### Example 9: Rate of Conversion of Sucrose to FOS

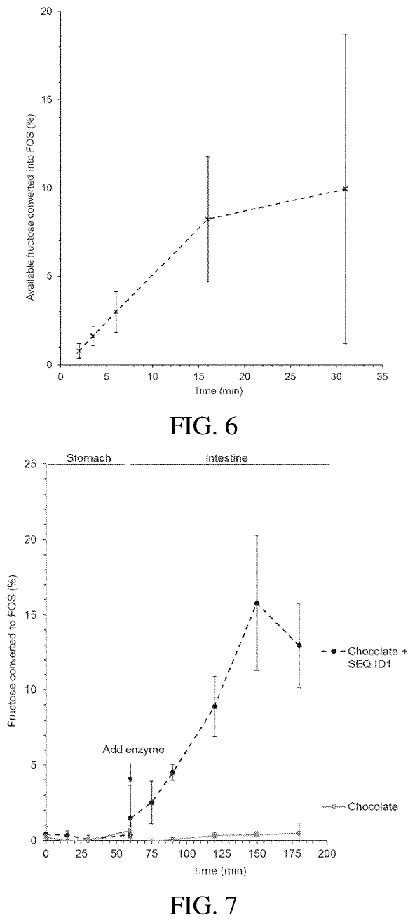

The speed of sucrose conversion into FOS was tested with 10 μg/mL SEQ ID NO 1 in simulated duodenal conditions (Houghton et al. Food Chemistry. 2014 15; 151:352-7) with 125 mM sucrose at 37° C. When all time points finished, the reaction was stopped, and free glucose/fructose was determined to infer FOS production.
Results are shown in FIG. 6. FOS production appeared to be linear in the first 16 min. 8.2±3.5% of available fructose was converted to FOS within 16 min, where 15-30 mins is a physiologically relevant timeframe for sucrose absorption in the small intestines.
This example confirms that significant sucrose conversion can be achieved using the FTases described herein, even at low FTase concentrations. Conversion is rapid and occurs within a physiologically-relevant timeframe.

### Example 10: Sucrose Conversion from Commercially Available Chocolate Bar

The performance of FTase with a commercially available chocolate bar (Cadbury's Dairy Milk) was tested in a dynamic gut model (Houghton et al. Food Chemistry. 2014 15; 151:352-7). The gut model was run at 50% of the original methodology (final complete volume=95 mL) and at 37° C. The digest was mixed with an overhead stirrer. Half a serving (22.5 g, containing ˜11.3 g sucrose) chocolate was cubed and mixed with synthetic saliva before adding to resting synthetic gastric fluid (total volume=35 mL). Synthetic gastric fluid includes 0.5 mg/mL pepsin and 0.04 mg/mL gastric lipase. A peristaltic pump was used to add secretions at constant rate. Gastric secretions were added at 0.25 mL/min for 1 h (total volume=50 mL). 12.5 mL fresh porcine bile was added to the digest followed by 475 μg of SEQ ID NO 1 (concentration at full gut volume=5 μg/mL). Control reactions with an equivalent volume of water to SEQ ID NO 1 were run in parallel. Synthetic pancreatic secretions including 7 mg/mL pancreatin were added at 0.25 mg/mL for 2 h (final total volume=95 mL). At each time point samples were taken an inactivated before determining free glucose/fructose as described previously to determine the quantity of available fructose converted into FOS.
Results are shown in FIG. 7. 2.5±1.5% of available fructose was converted into FOS in min and 4.5±0.5% in 30 min. Conversion of sucrose continued for at least 90 min with ±4.5% of available fructose incorporated into FOS in 90 min.
This example confirms that the FTase enzymes described herein are capable of converting significant sucrose to FOS in physiologically- and commercially-relevant compositions including in the presence of fats and lipids, other carbohydrates, and other food particles without being inhibited by such components, even at low FTase concentrations.

### Details of the Sequence Listing

SEQ ID NO: 1 shows the amino acid sequence of the fructosyltransferase of gene inuGB from Lactobacillus gasseri DSM 20604. Some or all of the residues shown in grey/bold/bold&underlined are believed to be associated with the active site of the protein.
SEQ ID NO: 2 shows the amino acid sequence of the fructosyltransferase of gene inuGA from Lactobacillus gasseri DSM 20243. Some or all of the residues shown in grey/bold/bold&underlined are believed to be associated with the active site of the protein.
SEQ ID NO: 3 shows the amino acid sequence of the fructosyltransferase of gene inuJ from Lactobacillus johnsonii NCC 553. Some or all of the residues shown in grey/bold/bold&underlined are believed to be associated with the active site of the protein.
SEQ ID NO: 4 shows the amino acid sequence of the fructosyltransferase of gene inu from Lactobacillus reuteri 121, L. reuteri TMW1.106. Some or all of the residues shown in grey/bold/bold&underlined are believed to be associated with the active site of the protein.
SEQ ID NO: 5 shows the amino acid sequence of the fructosyltransferase of gene inuO from Bacillus agaradhaerens. Some or all of the residues shown in grey/bold/bold&underlined are believed to be associated with the active site of the protein.
SEQ ID NO: 6 shows the amino acid sequence of the fructosyltransferase of gene inu from Lactobacillus reuteri TMW1.106. Some or all of the residues shown in grey/bold/bold&underlined are believed to be associated with the active site of the protein.
SEQ ID NO: 7 shows the amino acid sequence of the fructosyltransferase of gene AaFT32A from Aspergillus acleatus. Some or all of the residues shown in grey/bold/bold&underlined are believed to be associated with the active site of the protein.
SEQ ID NO: 8 shows the amino acid sequence of the fructosyltransferase of gene sft from Aspergillus sydowii. Some or all of the residues shown in grey/bold/bold&underlined are believed to be associated with the active site of the protein.
SEQ ID NO: 9 shows the amino acid sequence of the fructosyltransferase e of gene sacB from Bacillus amyloliquefaciens DSM 7=ATCC 23350. Some or all of the residues shown in grey/bold/bold&underlined are believed to be associated with the active site of the protein.
SEQ ID NO: 10 shows the amino acid sequence of the fructosyltransferase of gene sacB K315A from B. megaterium DSM319. Some or all of the residues shown in grey/bold/bold&underlined are believed to be associated with the active site of the protein.

SEQ ID NO: 1
AVKQDEKAAT AVKANTEVKA NETSTKSASK DNKAELKGQI KDIVKESGVD TSKLTDDQIN 60
ELNKISFSKE AKSGTQLTYS DEKKIAKTLI EQDARYAVPF FNASKIKNMP AAKTLDAQTG 120
KVEDLEIWDS WPVQDAKTGY VSNWNGYQLV IGMMGVPNTN DNHIYLLYNK YGDNNENNWK 180

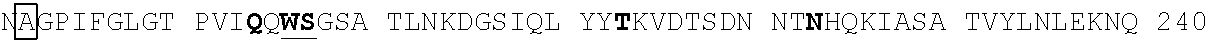

DKISIAHVDN DHIVFEGDGY HYQTYNQWKK TNKGADNIAM RDAHVIDDKD GNRYLVFEAS 300
TGTENYQGAD QIYQWLNYGG TNKDNLGDEL QILSNSDIKD RAKWSNAAIG IIKLNNDTKN 360
PGVEKVYTPL ISAPMVSDEI ERPDVVRLGN KYYLFAATRL NRGSNDDAWM AANKAVGDNV 420
AMIGYVSDNL THGYVPLNES GVVLTASVPA NWRTATYSYY AVPVEGRDDQ LLITSYITNR 480
GEVAGKGMHA TWAPSFLLQI NPDNTTTVLA KMTNQGDWIW DDSSENADMM GVLEKDAPNS 540
AALPGEWGKP VDWDLIGGYN LKPHQ 565
SEQ ID NO: 2
DAVKQDEKAA TSFKTNTEEK ANETSTKTAS NDNKAELKGQ IKDIVKESDV DTSKLINDQI 60
NELNKINFSK EAKSGTQLTY SDFKKIAKTL IEQDARYAIP FFNASKIKNM PAAKTMDAQT 120
GKVEDLEIWD SWPVQDAKTG YVSNWNGYQL VVGMMGVENT NDNHIYLLYN KYGDNNENNW 180

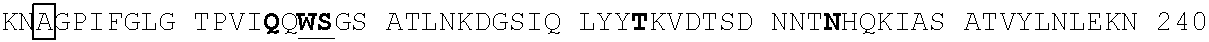

QDKISIAHVD NDHIVFEGDG YHYQTYNQWK KTNKGADNIA MRDAHVIDDK DGNRYLVFEA 300
STGTENYQGA DQIYQWLNYG GINKDNLGDF FQILSNSDIK DRAKWSNAAI GIIKLNNDTK 360
NPGVEKVYTP FISSPMVSDE IERPDVVRLG NKYYLFAATR LNRGSNDDAW MAANKAVGDN 420
VAMIGYVSDN LTHGYVPLNE SGVVLTASVP ANWRTATYSY YAVPVEGRDD QLLITSYITN 480
RGEVAGKGMH ATWAPSELLQ INPDNTTTVL AKMTNQGDWI WDDTSENDDM MGVLKKDAPN 540
SAALPGEWGK PVDWDLIGGY NLKPHQP 567
SEQ ID NO: 3
DDVKQVEKKD SVDKTNAEEN KDSSVKPAEN ATKAELKGQV KDIVEESGVD TSKLINDQIN 60
ELNKINESKE AKSGTQLTYN DEKKIAKTLI EQDARYAIPF FNASKIKNMP AAKTLDAQSG 120
KVEDLEIWDS WPVQDAKTGY VSNWNGYQLV IGMMGVPNVN DNHIYLLYNK YGDNDENHWK 180

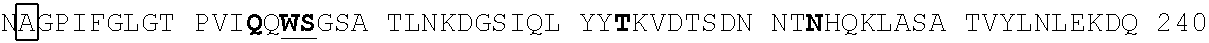

DKISIAHVDN DHIVFEGDGY HYQTYDQWKE TNKGADNIAM RDAHVIDDDN GNRYLVFEAS 300
TGTENYQGDD QIYQWLNYGG TNKDNLGDFF QILSNSDIKD RAKWSNAAIG IIKLNDDVKN 360
PSVAKVYSPL ISAPMVSDEI ERPDVVKLGN KYYLFAATRL NRGSNDDAWM ATNKAVGDNV 420
AMIGYVSDNL THGYVPLNES GVVLTASVPA NWRTATYSYY AVPVEGRDDQ LLITSYITNR 480
GEVAGKGMHA TWAPSFLLQI NPDNTTTVLA KMTNQGDWIW DDSSENPDMM GVLEKDAPNS 540
AALPGEWGKP VDWDLIGGYN LKPHQP 566
SEQ ID NO: 4
DTNIENNDSS TVQVITGDND IAVKSVTLGS GQVSAASDTT IRTSANANSA SSAANTQNSN 60
SQVASSAAIT SSTSSAASSN NTDSKAAQEN TNTAKNDDTQ KAAPANESSE AKNEPAVNVN 120
DSSAAKNDDQ QSSKKNTTAK LNKDAENVVK KAGIDPNSLT DDQIKALNKM NFSKAAKSGT 180
QMTYNDFQKI ADTLIKQDGR YTVPFFKASE IKNMPAATTK DAQTNTIEPL DVWDSWPVQD 240

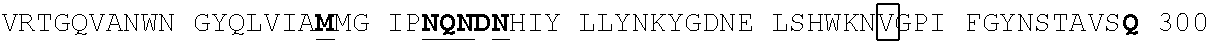

EWSGSAVLNS DNSIQLFYTR VDTSDNNINH QKIASATLYL TDNNGNVSLA QVANDHIVFE 360
GDGYYYQTYD QWKATNKGAD NIAMRDAHVI EDDNGDRYLV FEASTGLENY QGEDQIYNWL 420
NYGGDDAFNI KSLFRILSND DIKSRATWAN AAIGILKLNK DEKNPKVAEL YSPLISAPMV 480
SDEIERPNVV KLGNKYYLFA ATRINRGSND DAWMNANYAV GDNVAMVGYV ADSLTGSYKP 540
LNDSGVVLTA SVPANWRTAT YSYYAVPVAG KDDQVLVTSY MTNRNGVAGK GMDSTWAPSF 600
LLQINPDNTT TVLAKMTNQG DWIWDDSSEN LDMIGDLDSA ALPGERDKPV DWDLIGYGLK 660
PHD 663
SEQ ID NO: 5
TSDWDAEDDY TAVWTRQQAE NVALTKDTTA PLLETDEDFE LVAPDKWVWD TWPLQNRDGS 60
LAQVNGYTIA FALVAPRDLG WGERHTEARI GMFYSKDGKD WTYAGIPYDY DKAYGHMQWA 120
GSAMLDKDGK VHFFYTATGR KDNSEYEDQP GWEPMAEQRL AKTTEDISAD KDGVHLTKED 180
EHQIMLEADG EYYETLGQWG SNGNIISAFR DPFFFQDPNT GEEYIIWEGQ AGPKSNGLKP 240
ENIGDEAYRK NANVPDRAEL YNGNIGIAKV LDEDVSELKM LPPLLESIGV NHQLERPHVV 300
VDGDTYYLLT ISHTFTYAPG LIGPEGLYGF VNEGGLRGDY EPLNDGGLVI GNPAESPGQA 360
YSWWVAPDGQ VISFINEPLD ENGEVQFVGT FAPTLQLSED GDQTKIEKEM GYGEIRPFGA 420
YR 422
SEQ ID NO: 6
DTNTENNDSS TVHVTTGDND IAVKSAILGS GQVSAASDAT IKNSANANSA SSAANTQNSN 60
SQVASSAATT SSTSSAASSN NTDSKAAQEN ANTAKNDDTQ KAAPANESSE AKNEPAVNVN 120
DSSAAKNDDQ QSSKKNTTAK LNKDAENVVK KAGIDPNSLT DDQIKALNKM NESKAAKSGT 180
QMTYNDFQKI ADTLIKQDGR YTVPFFKASE IKNMPAATTK DAQTNTIEPL DVWDSWPVQD 240

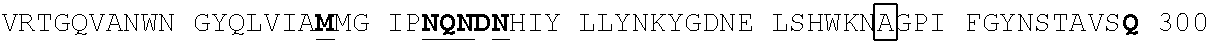

EWSGSAVLNS DNSIQLFYTR VDTSDNNTNH QKIASATLYL TDNNGNVSLA QVANDHIVFE 360
GDGYYYQTYD QWKATNKGAD NIAMRDAHVI EDDNGDRYLV FEASTGLENY QGENQIYNWL 420
NYGGDDAFNI KSLFRILSND DIKSRATWAN AAIGILKLNK DEKNPKVAEL YSPLISAPMV 480
SDEIERPNVV KLGNKYYLFA ATRLNRGSND DTWMNANYAV GDNVAMVGYV ADSLTGSYKP 540
LNDSGVVLTA SVPANWRTAT YSYYAVPVAG KDDQVLVTSY MTNRNGVAGK GMDSTWAPSF 600
LLQINQDNTT TVLAKMTNQG DWIWDDSSEN LDMIGDLDSA ALPGERDKPV DWDLIGYGLK 660
PHD 663
SEQ ID NO: 7
SYHLDTTAPP PTNLSTLPNN TLFHVWRPRA HILPAEGQIG DPCABYTDPS TGLFHVGFLH 60
DGDGIAGATT ANLATYTDTS DNGSFLIQPG GKNDPVAVFD GAVIPVGVNN TPTLLYTSVS 120
FLPIHWSIPY TRGSETQSLA VARDGGRRED KLDQGPVIAD HPFAVDVTAF RDPEVERSAR 180
LDVLLSLDEE VARNETAVQQ AVDGWTEKNA PWYVAVSGGV HGVGPAQFLY RQNGGNASEF 240
QYWEYLGEWW QEATNSSWGD EGTWAGRWGF NFETGNVLFL TEEGHDPQTG EVFVTLGTEG 300
SGLPIVPQVS SIHDMLWAAG EVGVGSEQEG AKVEFSPSMA GFLDWGFSAY AAAGKVLPAS 360
SAVSKTSGVE VDRYVSFVWL TGDQYEQADG FPTAQQGWTG SLLLPRELKV QTVENVVDNE 420
LVREEGVSWV VGESDNQTAT LRTLGITIAR ETKAALLANG SVTAEEDRTL QTAAVVPFAQ 480
SPSSKFFVLT AQLEFPASAR SSPLQSGFEI LASELERTAI YYQFSNESLV VDRSQTSAAA 540
PTNPGLDSFT ESGKLRLFDV IENGQEQVET LDLTVVVDNA VVEVYANGRF ALSTWARSWY 600
DNSTQIRFFH NGEGEVQFRN VSVSEGLYNA WPERN 635
SEQ ID NO: 8
MKLPSSLDIL LARQAVGGTE VDYDSPPPDL TTLPENSLFE TWRPKIHVLP PNGQIGDPCA 60
HYNDPATGLF HVGFLHNGTG ISSVYTDDLV TYRDINPNGG YIIVAGGPND PEAVFDGSVI 120
PSGIDDLPTL LYTSVTSLPI HWTLPYTPGS ETQSLAVSDD GGHHFDKLDR GPVIPLPPDG 180
LDVTAFRDPY VFQNHEVDEV TGSDPDTWYA AISGGVHDVG PGIFLYRNQD SSFENWEYLG 240
EWWQEPANST WGDGTWAKRW GYNFETGNVF SLDREGYNVD GHTEMTIGVE GAYAPIQPSV 300
TSMHAMLWAA GNVSSENGEN VTFTPYMAGA LDWGMAAYAG AGKVLPSTSQ ASEKSGAPDR 360
FISWVWLTGD EFGAAAGFPA AQQGWQNTLL LPRELSIHTI QNVVDNELIH ETASWRVAEH 420
GGERRSGGVE LETIGINIAR ETYDAIVSSG TSFEEPSRDI NESGTIPFER SPTSRFFALE 480
AQISFPQSAR DSEVQSGFQI LASELEWTTI YYQFSNESIV IDRNHTSAAS ETTPGLGTVT 540
ESGRIRLFDI AGGCDHDGHG GHDGGNDDDH NGDGDHSGDG DHNDDDDHNV DGDDKERARY 600
QKRDGPCDKD HDKVETLDLT IVVDNSVLEV YANSREVVST WVRPWYTNST EIRFFHNGEG 660
EVSFDNIAVH DGLYDAYPDR DN 682
SEQ ID NO: 9
KENNQKAYKE TYGVSHITRH DMLQIPKQQQ NEKYQVPQED QSTIKNIESA KGLDVWDSWP 60
LQNADGTVAE YNGYHVVFAL AGSPKDADDT SIYMFYQKVG DNSIDSWKNA GRVFKDSDKF 120
DANDPILKDQ TQEWSGSATF TSDGKIRLFY TDYSGKHYGK QSLTTAQVNV SKSDDTLKIN 180
GVEDHKTIFD GDGKTYQNVQ QFIDEGNYTS GDNSTERDPH YVEDKGHKYL VFEANTGTEN 240
GYQGEESLEN KAYYGGGTNF FRKESQKLQQ SAKKRDAELA NGALGIIELN NDYTLKKVMK 300
PLITSNTVTD EIERANVEKM NGKWYLFTDS RGSKMTIDGI NSNDIYMLGY VSNSLTGPYK 360
PLNKTGLVLQ MGLDPNDVTF TYSHFAVPQA KGNNVVITSY MTNRGFFEDK KATFGPSFLM 420
NIKGNKTSVV KNSILEQGQL TVN 443
SEQ ID NO: 10
KGNDSKDFNN SYGISHITRD NMVKIPQQQN SDQFKVPAFD ESTIKNIASA KGKNASGNTI 60
DLDVWDSWPL QNADGTVATY HGYQIVFALA GDPKDSNDTS VYLFYKKAGD KSIDSWKNAG 120
RVFKDSDKFV PNDPHLKNQT QEWSGSGTLT KDGKVRLFYT DYSGKQYGKQ TLTTAQVNMS 180
QPNDNTLKVD GVEDYKSIFD GDGKIYQTVQ QFIDEGGYDT GDNHTLRDPH YIEDNGHKYL 240
VFEANTGTED GYQGEDSLYN RAYYGGNNPF FQSEKKKLLE GSNKEKASLA NGALGIIELN 300
DDYTLKKVMK PLITSNTVTD EIERANIFKK DGKWYLFTDS RGSKMTIDGI GQDDVYMLGY 360
VSNTLTGKYK PLNDTGLVLH MDLDPNDKTF TYSSFAVPQT KGDNVVITSY MTNRGFYEDN 420
HSTFAPSFLV NIDGSKTSVV KDRVLEQGQL TVDED

1. An in vivo method of reducing fructose uptake in a subject, the method comprising administering to the subject an isolated fructosyltransferase.

2. An in vivo method of reducing the formation of fructose via metabolism of sucrose in a subject, the method comprising administering to the subject an isolated fructosyltransferase.

3. An in vivo method of reducing glucose uptake and/or of reducing the formation of glucose via metabolism of sucrose in a subject, the method comprising administering to the subject an isolated fructosyltransferase.

4. A method according to any one of the preceding claims, which is a method of producing a fructooligosacharide in a subject in vivo, the method comprising administering to the subject an isolated fructosyltransferase and thereby converting sucrose to said fructooligosacharide

5. A method according to any one of the preceding claims, wherein said fructosyltransferase is an inulosucrase or a levansucrase.

6. A method according to any one of the preceding claims, wherein said fructosyltransferase is an inulosucrase of EC class 2.4.1.9.

7. A method according to any one of the preceding claims, which is a method of reducing fructose uptake in a subject and producing inulin in vivo, the method comprising administering to the subject an isolated inulosucrase and thereby converting sucrose to inulin in vivo.

8. A method according to any one of the preceding claims which is a method of reducing the formation of fructose via metabolism of sucrose in a subject and producing inulin in vivo, the method comprising administering to the subject an isolated inulosucrase and thereby converting sucrose to inulin in vivo.

9. A method according to any one claims 1 to 5, wherein said fructosyltransferase is a levansucrase of EC class 2.4.1.10.

10. A method according to any one of the preceding claims, wherein said fructosyltransferase comprises a polypeptide according to any one of SEQ ID NOs: 1 to 10 or a functional variant thereof.

11. A method according to any one of the preceding claims, wherein said fructosyltransferase has:
i) at least 70% homology to SEQ ID NO: 1, wherein said homology is assessed relative to positions 128, 129, 153, 158, 159, 160, 162, 196, 197, 281, 282, 298, 379, 381, 399, 402, 457, 458 and 480 of SEQ NO: 1; or
ii) at least 70% homology to SEQ ID NO: 5, wherein said homology is assessed relative to positions 49, 50, 73, 82, 83, 84, 85, 86, 119, 120, 209, 210, 293, 295 and 361 of SEQ ID NO: 5; or
iii) at least 70% homology to SEQ ID NO: 8, wherein said homology is assessed relative to positions 54, 55, 56, 57, 58, 59, 74, 75, 116, 265, 338, 339, 366, 370, 372 and 373 of SEQ ID NO: 8.

12. A method according to any one of the preceding claims, wherein said fructosyltransferase comprises alanine at the position corresponding to A182 of SEQ ID NO: 1.

13. A method according to any one of the preceding claims, wherein said fructosyltransferase comprises phenylalanine at the position corresponding to F372 of SEQ ID NO: 8 and/or comprises glycine at the position corresponding to G373 of SEQ ID NO: 8.

14. A method according to any one of the preceding claims, wherein said fructosyltransferase has a solubility GRAVY score of −0.4 or more negative than −0.4.

15. A method according to any one of the preceding claims, wherein said fructosyltransferase is derived from an organism of genus Lactobacillus, Bacillus, Leuconostoc, Streptomyces, Aspergillus, or Clostridium.

16. A method according to any one of the preceding claims, wherein said fructosyltransferase is derived from an organism of species Lactobacillus gasseri, Lactobacillus johnsonii, Lactobacillus reuteri, Bacillus agaradhaerens, Bacillus amyloliquefaciens, Bacillus megaterium, Bacillus subtilis, Leuconostoc citreum, Leuconostoc mesenteroides, Streptomyces viridochromogenes, Aspergillus acelatus, Aspergillus sydowii, or Clostridium acetobutylicum.

17. A method according to any one of the preceding claims, wherein said fructosyltransferase is expressed by or is obtainable by expression from an organism of genus Escherichia, Lactobacillus, Saccharomyces, Bacillus, Pichia, Trichoderma or Aspergillus; preferably E. coli, S. cerevisiae, B. subtilis, P. pastoris, T reesei, A. niger, or A. oryzae.

18. A method according to any one of the preceding claims, wherein the fructosyltransferase is comprised in a nutraceutical composition comprising said fructosyltransferase and one or more nutraceutically acceptable filler, stabilizing agent, coloring agent or flavouring agent.

19. A method according to claim 18, wherein said nutraceutical composition is formulated as a tablet, a troche, a lozenge, an aqueous or oily suspension, a dispersible powder or as granules.

20. A method according to any one of the preceding claims, comprising orally administering said fructosyltransferase or said nutraceutical composition to said subject.

21. A method according to any one of the preceding claims, wherein said method is a non-therapeutic method; preferably wherein said method does not comprise the treatment of the human or animal body by therapy or surgery.

22. An isolated fructosyltransferase for use in
i) reducing fructose uptake in a subject,
ii) reducing the formation of fructose via metabolism of sucrose in a subject;
iii) reducing glucose uptake and/or of reducing the formation of glucose via metabolism of sucrose in a subject;
iv) producing a fructooligosacharide in a subject, said use comprising administering to the subject an isolated fructosyltransferase and thereby converting sucrose to said fructooligosacharide;

23. An isolated fructosyltransferase for use according to claim 22, wherein:
i) said isolated fructosyltransferase is for use in reducing fructose uptake and producing inulin in the subject, and said use comprises administering to the subject the isolated inulosucrase and thereby converting sucrose to inulin in vivo; or
ii) said isolated fructosyltransferase is for use in reducing the formation of fructose via metabolism of sucrose in the subject and producing inulin in vivo, and said use comprises administering to the subject the isolated inulosucrase and thereby converting sucrose to inulin in vivo.

24. An isolated fructosyltransferase for use according to claim 22 or 23 wherein said fructosyltransferase is as defined in any one of claims 5, 6, or 9 to 19.

25. An isolated fructosyltransferase for use according to any one of claims 21 to 24, wherein said use comprises orally administering said isolated fructosyltransferase to said subject;
wherein said use optionally comprises orally administering said isolated fructosyltransferase to said subject in the form of a pharmaceutically acceptable composition or a nutraceutically acceptable composition.

26. A nutraceutical composition comprising an isolated fructosyltransferase and one or more nutraceutically acceptable filler, stabilizing agent, colouring agent or flavouring agent.

27. A composition according to claim 26, wherein said composition is a dietary supplement.

28. A pharmaceutically acceptable composition comprising an isolated fructosyltransferase and one or more pharmaceutically acceptable carrier, excipient, or diluent.

29. A composition according to any one of claims 26 to 28, wherein the isolated fructosyltransferase is as defined in any one of claims 4 to 17.

30. A composition according to any one of claims 26 to 29, wherein said composition is for oral administration; preferably wherein said composition (i) comprises an enteric coating and/or (ii) is formulated as a tablet, a troche, a lozenge, an aqueous or oily suspension, a dispersible powder or as granules.

31. A composition according to any one of claims 26 to 30 for use in medicine.

32. A food composition or foodstuff comprising an isolated fructosyltransferase and one or more carbohydrate, fat, lipid, flavouring agent, or colouring agent.

33. A food composition or foodstuff according to claim 32, comprising sucrose.

34. A composition according to claim 32 or 33, wherein the isolated fructosyltransferase is as defined in any one of claims 4 to 17.

35. A method of suppressing a subject's appetite and/or increasing a subject's satiety, comprising administering to the subject an isolated fructosyltransferase or a composition according to any one of claims 26 to 34.

36. A method according to claim 35, wherein the isolated fructosyltransferase is as defined in any one of claims 4 to 19.

37. A method according to claim 35 or 36, wherein said method is a non-therapeutic method; preferably wherein said method does not comprise the treatment of the human or animal body by therapy or surgery.

38. An isolated fructosyltransferase, optionally as defined in any one of claims 4 to 19, or a pharmaceutically acceptable composition according to any one of claims 28 to 30, for use in treating or preventing metabolic syndrome, diabetes, non-alcoholic fatty liver disease or constipation in a subject in need thereof.

39. An isolated fructosyltransferase, optionally as defined in any one of claims 4 to 19, or a pharmaceutically acceptable composition according to any one of claims 28 to 30, for use in treating or preventing obesity in a subject in need thereof.

40. A method according to any one of claims 35 to 37, comprising orally administering said isolated fructosyltransferase or composition to said subject.

41. An isolated fructosyltransferase or pharmaceutically acceptable composition for use according to claim 38 or 39, wherein said use comprises orally administering said isolated fructosyltransferase or said pharmaceutically acceptable composition to said subject.
